(19) United States
(12) Patent Application Publication
(10) Pub. No.: US 2002/0072097 A1
(43) Pub. Date: Jun. 13, 2002

(54) Molecular breeding of transposable elements

(76) Inventors: Stephen delCardayre, Belmont, CA (US); Ranjan Patnaik, San Jose, CA (US); Phillip Patten, Menlo Park, CA (US); Matthew Tobin, San Jose, CA (US); Jon E. Ness, Sunnyvale, CA (US); Anthony Cox, Mountain View, CA (US); Lorraine J. Giver, Santa Clara, CA (US); Kevin McBride, Davis, CA (US); Kenneth Zahn, Redwood City, CA (US)

Correspondence Address: LAW OFFICES OF JONATHAN ALAN QUINE, P O BOX 458, ALAMEDA, CA, 94501

(21) Appl. No.: 09/899,814

(22) Filed: Jul. 5, 2001

Related U.S. Application Data
(60) Provisional application No. 60/216,798, filed on Jul. 7, 2000.

(51) Int. Cl. C12P 19/34; C12N 15/87; C12N 15/74

(52) U.S. Cl. 435/91.2; 435/455; 435/473; 435/320.1

(57) ABSTRACT

Methods for producing transposable elements with improved properties as vectors are provided. Directed evolution procedures are employed to improve characteristics of transposable elements, including transposons and insertion sequences as vectors. Methods for generating diversity in vivo and in vitro using transposable elements as vectors are provided.

### CROSS REFERENCE TO RELATED APPLICATIONS

This application claims priority to and benefit of U.S. Provisional Application No. 60/216,798, filed Jul. 7, 2000, the specification of which is incorporated herein in its entirety for all purposes.

### BACKGROUND OF THE INVENTION

Industrial production of many biochemicals is currently achieved through use of whole cells as biocatalysts or by fermentation. Economic production of these chemicals are typically dependent on the productivity of the biocatalyst under process conditions, which generally tend to be significantly different than the conditions for which the biocatalyst has naturally evolved. The current technology used to engineer strains to be more productive under desired process conditions generally involves one or both of: various forms of mutagenesis on a host organism coupled with screens and selections and/or overexpression of desired enzymes using standard molecular biology tools.

Although the above methods are successful to a certain extent, many limitations and disadvantages exist. For example, classical mutagenesis and screening procedures are time consuming, and in most cases, improvements observed in one host cannot be transferred to another host due to lack of significant knowledge about the relevant genetic interactions in the host and recipient species. In cases where genetic methodology is used, only pair-wise recombination of useful mutations can be assessed at any one time. Briefly, the synergistic effect of many useful mutations on a desired phenotype cannot be assessed conveniently using current methods due to the difficulty in assessing the mutations in combinatorial fashion.

Typically, in a classical strain improvement program, many desirable phenotypes are observed in different host backgrounds but the ability to combine these phenotypes into a single production strain is severely limited due to lack of methodology for inter-species genetic exchange, low homologous recombination efficiency, low electroporation efficiency in certain cases and most importantly lack of a suitable method for creating combinatorial genomes.

The evolution of microbial genomes is catalyzed by the processes of horizontal gene transfer. Indeed, these processes most closely resemble the exchange of genetic information that occurs during the sexual cycle of eukaryotic organisms. Natural competence, general transduction, conjugation, and transposon mediated gene exchange all contribute to horizontal gene transfer. Insertion sequences and transposons are found distributed throughout most genomes thus far investigated. The mobilization of IS elements and transposons within and between genomes is a primary mechanism for the reorganization of genome structure and the horizontal exchange of genetic information.

The goal of rapidly evolving whole microbial cells by “whole genome shuffling” will most efficiently be realized when the natural mechanisms by which microbial cells evolve can be harnessed and accelerated in a laboratory setting. Described here is a general approach to microbial breeding that exploits the efficiency of transposons to mobilize and insert large pieces of heterologous DNA into the chromosome of a broad range of microbial hosts. This mechanism of genetic exchange employs non-homologous recombination and provides a means by which divergent heterologous DNA can be incorporated into the genome of an unrelated host. Extensive processes for whole genome shuffling are found in USSN 09/116,188 “Evolution of Whole Cells and Organisms by Recursive Recombination” by del Cardayre et al. filed Jul. 15, 1998 and PCT publications WO 00/04190 “Evolution of Whole Cells and Organisms by Recursive Sequence Recombination,” by del Cardayre et al. published Jan. 27, 2000. The present invention provides additional improvements in horizontal gene transfer vectors and artificial evolution methods.

### SUMMARY OF THE INVENTION

The present invention provides methods for producing transposable elements, including transposons and insertion sequences, with improved properties. In general, the methods of the invention involve diversifying, e.g., recombining, polynucleotide segments corresponding to one or more component of a transposable element to produce a library of recombinant transposable element components. The library is then evaluated to identify members with improved properties. Optionally, the process is performed in a recursive fashion. In some embodiments, the transposable element is recovered following transposition into the host cell.

For example, substrates for diversification, e.g., recombination, or “shuffling” reactions can include any component of a transposable element, such as a transposase or an inverted repeat. Alternatively, only a subsequence of such a component provides the basis for recombination. In other cases, multiple components, including entire transposable elements, e.g., mini-transposons, mini-IS elements, etc., are recombined, e.g., shuffled simultaneously. Suitable substrates for the methods of the present invention include transposable elements derived from a variety of sources, including bacterial, fungal, plant and animal transposable elements. Such transposable elements can be broadly categorized based on their mechanism of transposition into Class I, e.g., retrotransposons, retroposons, and SINE-like elements, e.g., Ty-1, Copia, gypsy, and the like, and Class II, e.g., Fot1/Pogo, Tc1/Mariner, etc. Both Class I and Class II transposable elements are substrates of the invention. In certain preferred embodiments, transposable elements that are TN3, TN5, TN10, TN917, ISS1, TN5990, Ty1, Ty2, Ty3, and mariner are substrates for the diversification, e.g., shuffling methods of the invention. Diversification, e.g., shuffling of the transposable element sequences is performed in vitro, in vivo, in silico, or any combination thereof.

The methods of the present invention are used to produce transposable elements with a variety of improved properties; in particular, with respect to their performance as delivery vectors. Desirable properties include: altered specificity of integration, host adaptation, increased or decreased recombinase activity, increased or decreased transposase activity, increased or decreased recombinase specificity, increased or decreased transposase specificity, increased or decreased size of exogenous DNA transposed, increased or decreased copy number, increased or decreased efficiency of transposition, increased or decreased preference for episomal targeting, increased or decreased preference for chromosomal targeting, increased efficiency of integration into non-supercoiled DNA, and increased efficiency of in vitro transposition.

In general, transposable elements, or their components with desired properties are identified by one or more selection or screening protocols. In one preferred embodiment, components of transposable elements that mediate in vitro transposition with increased efficiency are identified by evaluating in vitro transposition reactions comprising a transposase, a donor polynucleotide having an inverted repeat, and a target polynucleotide, of which one or more components results from diversification procedures, e.g., shuffling. In some embodiments, the in vitro transposition reactions include transposomes.

In another preferred embodiment, transposable elements that transpose with increased efficiency in a specified host cell type are identified by introducing a plurality of transposable elements, differing by at least one nucleotide, into a population of host cells, and selecting host cells that have integrated the transposable element into a chromosome or episome. Such methods are facilitated by the use of a transposable element including, in the direction of transcription: (a) a polynucleotide comprising a transcription regulatory sequence; (b) a 5′ splice donor site; (c) a first inverted repeat; (d) a 3′ splice acceptor site; (e) a polynucleotide encoding a transposase; (f) a polynucleotide encoding a selectable marker; and (g) a second inverted repeat. In some embodiments the transposase is transiently expressed preceding transposition. Following transposition, e.g., integration, host cells expressing a sufficient level of a marker, e.g., antibiotic resistance, encoded by the transposable element are selected. In certain embodiments, the selected host cells are mammalian cells. In some cases, the transposable element is a Mariner-like transposable element, having a Mariner transposase and Mariner inverted repeats.

In some embodiments, sequences comprising a transposable element are incorporated into a recombinant vector such as a recombinant episomal vector, e.g., a plasmid. In one embodiment, the vector is a delivery vector. The delivery vector has an origin of replication active in one or more cloning hosts, as well as a conditional origin of replication active in a selected target cell; at least one screenable or selectable marker, e.g., antibiotic resistance, toxicity resistance, conferred prototrophy; and a mini-transposon having inverted repeats flanking a multicloning site (MCS) and a transposase operably linked to a promoter active in the selected target cell. In certain preferred embodiments, the transposase is derived by a directed evolution process. In some embodiments, the sequences encoding the transposase are situated in close proximity to an end of the mini-transposon.

Such recombinant delivery vectors are also an aspect of the invention. Exemplary replication origins of the vectors include origins derived from: ColE1, pACYC, p15A, RK4, RK6, pCM595, pSa, pUB110, pE194, pG+, 2 micron circles, and artificial chromosomes. Temperature sensitive origins of replication favorable in the vectors of the present invention include pSA3, pE194, and pG+tm. Mini-transposons derived from transposons or insertion sequence elements including insertion sequences and their components including inverted repeats and transposases selected from among: IS1, IS2, IS3, IS4, IS5, IS6, IS10, IS21, IS30, IS50, IS91, IS150, IS161, IS186, IS200, IS903, IS3411, IssHO1, IS600, IS22, IS52, IS222, IS401, IS402, IS403, IS404, IS405, IS411, IS476, IS60, IS66, IS426, IS492, IS4400, ISR1, ISRm1, ISRm2, RSRj-alpha, RSRj-beta, IS701, IS 231, IS2150, IS256, IS431, IS257, ISS1, IS110, IS466, ISL1, and Gamma delta, are favorably employed in the context of the present invention.

Similarly, transposons from a variety of sources including conjugative transposons, e.g., Tn916, Tn918, Tn919, Tn925, Tn1545, 3951, and BM6001 element; Class II transposons, e.g., Tn551, Tn917, Tn3871, Tn4430, Tn4556, Tn4451, Tn4452; and other transposons, e.g., Tn554, Tn3853; Tn4001, Tn3851, Tn552, Tn4002, Tn3852, Tn4201, and Tn4003 TN3, TN5, TN10, TN917, ISS1, TN5990, Ty1, Ty2, Ty3, and mariner are favorably employed as mini-transposons in the recombinant delivery vectors of the invention.

Transposable elements with improved characteristics are a feature of the present invention. Similarly, components, e.g., transpsosases, integrases, inverted repeats, etc., of transposable elements conferring improved characteristics are a feature of the invention. Transposable elements having (and transposable element components conferring) such desirable properties as altered specificity of integration, host adaptation, increased or decreased recombinase activity, increased or decreased transposase activity, increased or decreased recombinase specificity, increased or decreased transposase specificity, increased or decreased size of exogenous DNA transposed, increased or decreased copy number, increased or decreased efficiency of transposition, increased or decreased preference for episomal targeting, increased or decreased preference for chromosomal targeting, increased efficiency of integration into non-supercoiled DNA, and increased efficiency of in vitro transposition are produced by the methods of the invention.

In another aspect, the invention provides methods for producing a transposase that efficiently catalyzes in vitro tranposition. A population of polynucleotide segments encoding one or more transposases or subportions of one or more transposase are recombined to produce a library of variant transposases. The variant transposases are then evaluated for their ability to efficiently catalyze in vitro transposition. In an embodiment, variant transposases that efficiently catalyze in vitro transposition are identified by incubating a plurality of in vitro transposition reactions under conditions permissive for in vitro transposition, and identifying those reactions that proceed with greater efficiency than an in vitro transposition reaction mediated by a parental transposase. In vitro transposition reactions include: a variant transposase encoded by a member of the library of recombinant polynucleotides; a donor polynucleotide with at least one inverted repeat (e.g., one, two or a number sufficient for transposition); and a target polynucleotide. Transposases produced according to the methods are also a feature of the invention. In preferred embodiments, the transposases are derived by a directed evolution process from transposases of one or more of TN3, TN5, TN10, TN917, TN5990, ISS1, Ty1, Ty2, Ty3 and mariner. Similarly, reaction mixes and cells including the transposases produced by the methods of the invention are an aspect of the invention.

Another aspect of the invention relates to the generation of diversity in a population of nucleic acids. The invention provides methods of generating diversity in a population of nucleic acids by contacting a recombinant, e.g., shuffled transposable element, or a shuffled component of a transposable element with a plurality of subject nucleic acids under conditions permissive for transposition. Alternative embodiments involve contacting the transposable element, or transposable element component, and the subject nucleic acids in vitro or in vivo. In one embodiment, altered subject nucleic acids are identified.

In some embodiments, the recombinant, e.g., shuffled transposable element component is a transposase. In an embodiment, a transposome made up of a recombinant, e.g., shuffled transposase bound to a donor nucleic acid having sequences recognized by the shuffled transposase is introduced into a cell, e.g., by electroporation. In alternative embodiments, the transposome is contacted with the subject nucleic acids in an acellular reaction mix.

In another aspect, the invention provides methods for generating diversity in a population of nucleic acids in vitro using transposomes. Transposomes incorporating a diverse (e.g., from multiple species or strains of microorganism) library of donor nucleic acids having transposase recognition sites are recombined in vitro with a population of acceptor nucleic acids. Optionally, the recombinant nucleic acids are introduced into cells and cells expressing a desired phenotype is screened or selected. In some embodiments, the recombination process is performed recursively, with or without intervening screening or selection steps.

The invention further provides methods for identifying chromosomal loci that generate a desired level of gene expression. Generally, such methods involve (i) transfecting a plurality of host cells expressing a transposase with a vector characterized by inverted repeats flanking a promoter, a site specific recombinase recognition site, and one or more screenable or selectable marker; (ii) selecting host cells that have integrated the vector and express a sufficient level of a selectable marker encoded by the vector to survive selection; and (iii) evaluating the surviving host cells for a desired level of expression of a marker. Such vectors are a feature of the invention. For example, in the case of identifying a locus in a chromosome of a selected mammalian cell line expressing, e.g., a Mariner transposase, the inverted repeats of the vector are preferably derived from a transposable element, e.g., Mariner, the site specific recombinase recognition site comprises a loxP site, and the promoter comprises, e.g., a cytomegalovirus (CMV) promoter active in the selected cell line.

In preferred embodiments, the transposase is a recombinant, e.g., shuffled transposase with at least one improved property, e.g., sequence specificity, activity level, species selectivity, allostery, control, etc., relative to a parental transposase from which it is derived. In some embodiments, the vector also supplies expression of the transposase by including a polynucleotide encoding the transposase operably linked to a promoter functional in the host cells. Alternatively, the transposase activity is supplied by an additional vector, or integrated into a chromosome. In some embodiments, the transposase is transiently, e.g., inducibly, expressed. In some cases, a polynucleotide of interest is integrated into the chromosomal locus previously identified and integrants are identified exhibiting a desired level of expression of the gene of interest.

The present invention also provides, e.g., a transposable element comprising, in the order of transcription: an int encoding sequence and an xis encoding sequence, each operably linked to a promoter functional in the target cell; a mini-IS element; an origin of replication functional in a cloning host, a first and a second selectable marker; and a second, temperature sensitive, origin of replication functional in the target cell, is a feature of the invention.

### BRIEF DESCRIPTION OF THE DRAWING

FIGS. 1A-1C are schematic illustrations of recombinant vectors incorporating transposable elements.

FIGS. 2A-2B are schematic illustrations of transposon vectors.

FIG. 3 is a schematic illustration of a continuous fermentation protocol for selecting variants with a desired phenotype.

FIGS. 4A-4D schematically illustrate in vitro transposome mediated recombination.

### DETAILED DISCUSSION OF THE INVENTION

The present invention relates to the production of transposable elements with improved characteristics, most particularly, with respect to their function as vectors for genetic manipulation. Nucleic acid diversification procedures, such as shuffling are used to recombine and/or mutate naturally occuring, mutant and/or artificial polynucleotides corresponding to transposable elements and their components, e.g., repeat sequences, transposases, regulatory sequences and the like. Following generation of a library of recombinant transposable element sequences, transposable elements and transposable element components that exhibit desired properties are identified through a variety of screening and selection procedures. Transposable elements with novel and enhanced properties are valuable as vectors for delivering DNA into cells, and for generating diversity within a population of cells by transposition mediated events. In addition, isolated components, e.g., transposases are valuable as tools for mediating DNA delivery and recombination both in vitro and in vivo.

### DEFINITIONS

Unless defined otherwise, all scientific and technical terms are understood to have the same meaning as commonly used in the art to which they pertain. For the purpose of the present invention the following terms are defined below.

A “transposable element” (TE) or “transposable genetic element” is a DNA sequence that can move from one location to another in a cell. Movement of a transposable element can occur from episome to episome, from episome to chromosome, from chromosome to chromosome, or from chromosome to episome. Transposable elements are characterized by the presence of inverted repeat sequences at their termini. Mobilization is mediated enzymatically by a “transposase.”

Structurally, a transposable element is categorized as a “transposon,” (“TN”) or an “insertion sequence element,” (IS element) based on the presence or absence, respectively, of genetic sequences in addition to those necessary for mobilization of the element. A mini-transposon or mini-IS element lacks sequences encoding a transposase.

In the context of the present invention, a “component” of a transposable element refers to any identifiable functional unit, e.g., polynucleotide repeats, transposase, whether nucleic acid or protein, of a transposable element. A “subportion” of a transposable element or transposable element component refers to any subsequence of a transposable element or transposable element homolog, including artificial sequences, up to and including an entire transposable element or transposable element component.

A “parental” transposable element or transposable element component, e.g., transposase, refers to a transposable element, or component, that is provided as a substrate for a directed evolution process, e.g., nucleic acid shuffling, according to any of the formats described herein. Typically, such a substrate is provided in actual (e.g., in vitro, in vivo shuffling) or virtual (e.g., in silico shuffling) form as a polynucleotide “segment.”

An “in vitro transposition reaction” is a recombination between nucleic acid substrates, e.g., a donor DNA molecule and a target DNA molecule, mediated by a transposase in an acellular reaction mixture. The term “transposome,” or “synaptic complex,” refers to a functional complex made up of a transposase associated with a transposable polynucleotide via specific recognition sequences, e.g., inverted repeat sequences.

“Screening” is, in general, a two-step process in which one first determines which cells, organisms or molecules, do and do not express a detectable marker, or phenotype (or a selected level of marker or phenotype), and then physically separates the cells, organisms or molecules, having the desired property. “Selection” is a form of screening in which identification and physical separation are achieved simultaneously by expression of a selectable marker, which under some circumstances, allows cells expressing the marker to survive while other cells die (or vice versa). Screening reporters include visible markers such as luciferase, β-glucuronidase, green fluorescent protein (GFP) as well as functional attributes evaluated according to a variety of specific assays. Selectable markers include antibiotic and herbicide resistance genes. A special class of selectable markers are negatively selectable markers. Cells or organisms expressing a negatively selectable marker die under appropriate selection conditions while organisms lacking or having a non-functional form of the marker survive.

The present invention provides methods, characterized as artificial or directed evolution, for evolving transposable elements and components thereof to acquire desired properties. Directed evolution involves the generation of sequence diversity in a nucleic acid, or population of nucleic acids, followed by or interspersed with screening or selection procedures to identify nucleic acids with desired structural or functional properties or characteristics. The invention utilizes, e.g., MolecularBreeding™ technologies, in a process of directed evolution, to generate and optimize mutations resulting in transposable elements with improved characteristics, e.g., as vectors and mutagenic agents. The resultant transposable elements and components, e.g., transposases, are used to introduce and/or mobilize polynucleotides into or within a genome in a wide variety of applications.

In a general format, polynucleotide segments corresponding to a transposable element or a component of a transposable element, or to a subportion thereof, are recombined, in vitro, in vivo, or in silico to produce a library of recombinant transposable element polynucleotides. The polynucleotide segments provided can be physical, such as isolated DNAs derived from naturally occurring transposable elements or synthesized oligonucleotides corresponding to (or complementary to) a portion of a wild type or variant transposable element or component thereof. Alternatively, the polynucleotide segments can be virtual, e.g., in silico representations of a naturally occurring or synthetic DNA sequence stored in a computer readable medium.

The polynucleotide segments are recombined, and optionally mutated, one or more times to generate a library of recombinant transposable element polynucleotides. The recombination process can be performed in vitro, in vivo, or in silico, or in any combination of formats as described in further detail herein and in the cited references. The library is then evaluated, by a variety of techniques available in the art chosen to identify recombinants with the desired property.

For example, polynucleotide segments that are fragments derived by DNAse digestion from a transposable element isolated from a given bacterial or eukaryotic species can be combined in vitro with synthesized degenerate oligonucleotides corresponding to a variety of naturally occuring or artificial sequences, some or all or none of which correspond to sequences of known transposable elements. The segments are then recombined according to any of the procedures described herein, or in the cited references. For example, the DNAse generated segments described above can be recombined based on homology by PCR reassembly protocols previously described by the inventors and their coworkers.

Alternatively, in silico character strings representing polynucleotides of any number of transposable element and other sequences, e.g., recombinases, integrases, etc., can be recombined by a computer according to genetic algorithms that do not rely on homology. Optionally, the resulting recombinant polynucleotides can be synthesized, and if desired, subject to additional rounds of recombination in vitro or in vivo.

In some cases, the polynucleotide segments are recombined in the context of a recombinant vector. In other cases, individual components or transposable elements are recombined and subsequently recovered, e.g., by a polymerase chain reaction (PCR), ligase chain reaction (LCR), Qβ-replicase amplification, NASBA or cloning. Upon recovery, it is often desirable to conserve and/or reproduce the component or transposable element in the context of a vector.

Transposable elements, transposable element components and vectors comprising transposable elements, produced by the methods of the invention, are used to alter the genomes of cells and organisms both as mutagenic agents and as recombinant delivery vectors. In the former case, transposable elements with improved characteristics as mutagens, e.g., increased transposase activity, increased recombinase activity, decreased transposase specificity, decreased recombinase specificity, increased copy number, increased efficiency of transposition, etc., are introduced into cells where they are constitutively or inducibly activated to undergo transposition events. This provides the basis for novel and improved methods for generating diversity both in vitro and in vivo. In the latter case, transposable elements of the invention that are delivery vectors are employed to introduce sequences of interest into the genome of a cell (or organism). In addition, these methods are useful for the creation of combinatorial genomes.

Additionally, specialized vectors that include transposable elements and transposable element components useful for genetic manipulation are described. For example, vectors and methods useful for identifying a chromosomal locus capable of supporting a desired level of gene expression are provided, as are methods for integrating a gene of interest into such a locus.

### TRANSPOSABLE ELEMENTS

Transposable elements are DNA sequences that can move between locations within a genome, and in some cased between genomes. Transposable genetic elements have been identified in a wide range of organisms, including both prokaryotes and eukaryotes, and since their identification have found numerous uses as vectors, markers, and as mutagens. Transposable elements, as a group, share certain advantageous features that make them particularly well suited as agents of genetic change.

In general, transposable elements that include only sequences necessary for transposition are designated “insertion sequence (IS) elements,” or “insertion sequences.” IS elements contain genes encoding proteins necessary for transposition, (i.e., excision and insertion) flanked by short inverted repeats. In contrast, a “transposon” (TN) typically incorporates genetic sequences in addition to those involved in mobilizing the DNA. Often these additional sequences confer resistance to antibiotics or produce toxins. The conversion of an IS element to a transposon can occur when two IS elements surrounding a region of genomic DNA excise together mobilizing the intervening genomic DNA. Conjugal transposons further encode the ability to catalyze the conjugal transfer of the excised transposon to a different cell where it integrates into the chromosome.

Both IS elements and transposons are the subject of the present invention. IS elements can be readily adapted, e.g., as vectors for DNA delivery, through the introduction of a multiple-cloning site (MCS). Similarly, DNA sequences, e.g., genes of interest, can be engineered into transposons either as replacements for, or in addition to, sequences non-essential for mobilizing the transposon. Regardless of whether an IS element or transposon is selected, the transposable element can be manipulated according to the methods described herein to acquire novel and desirable properties.

Transposable elements can be categorized into two broad classes based on their mode of transposition. These are designated Class I and Class II; both have applications as mutagens and as delivery vectors, and both are subject to improvement by the methods of the invention. Class I transposable elements transpose by an RNA intermediate and use reverse transcriptases, i.e., they are retroelements. There are at least three types of Class I transposable elements.

Retrotransposons of the Ty-1/Copia family and the gypsy family. Retrotransposons typically contain LTRs, and genes encoding viral coat proteins (gag) and reverse transcriptase, RnaseH, integrase and polymerase (pol) genes.

Retroposons (LINE-like retroelements) have poly-A tails but do not have LTRs, and intact retroposons also contain gag and pol.

SINE-like elements are derived from transcripts of RNA polymerase III. They do not contain gag or pol or LTRs, and are trans-activated by RTs from the retroelements or retrotransposons.

Class II transposable elements transpose directly at the DNA level, and include the Fot1/Pogo or Tc1/Mariner families, among others. Class II transposons have short inverted repeats and often encode transposases of different types.

Transposition occurs by either a conservative or replicative mechanism depending on the transposable element.

So-called “Mini-transposons” lack transposases altogether, and can be constructed to permit provision of the transposase in trans.

Transposable elements are distributed throughout the genomes of a wide variety of species, including both prokaryotes and eukaryotes. Depending on the application, and in particular on the host cell to be the subject of manipulation by the transposable elements of the invention, a choice is made from among the myriad transposable elements.

### Bacterial Transposable Elements

Bacterial cells are especially amenable to genome manipulation, e.g., diversification, using transposable elements. Transposons and insertion sequences have been isolated and characterized from numerous gram-negative and gram-positive bacterial species, and bacterial TEs of both Class I and Class II varieties, and that are conjugative transposons are favorably employed in the methods of the invention. Of these, both insertion sequence elements and transposons have been cloned and characterized. Insertion sequences are typically between about 0.7 and 2 kb, while transposons range in size to greater than 50 kb. A number of references provide extensive lists of sources of sequences suitable in the context of the present invention (see, e.g., Galas and Chandler, Bacterial Insertion Sequences; Murphy, Transposable elements in gram-positive bacteria). The following are provided by way of illustration and not by limitation, as it will be readily understood that sequences derived or inferred from any transposable element, whether naturally occurring, mutant or artificial, can be recombined according to the methods of the invention to produce transposable elements with desired characteristics.

For example, insertion sequences and their components including inverted repeats and transposases selected from among: IS1, IS2, IS3, IS4, IS5, IS6, IS10, IS21, IS30, IS50, IS91, IS150, IS161, IS186, IS200, IS903, IS3411, IssHO1, IS600, IS22, IS52, IS222, IS401, IS402, IS403, IS404, IS405, IS411, IS476, IS60, IS66, IS426, IS492, IS4400, ISR1, ISRm1, ISRm2, RSRj-alpha, RSRj-beta, IS701, IS 231, IS2150, IS256, IS431, IS257, ISS1, IS110, IS466, ISL1, and Gamma delta, are all favorably employed in the context of the present invention.

Similarly, transposons from a variety of sources including conjugative transposons, e.g., Tn916, Tn918, Tn919, Tn925, Tn1545, 3951, and BM6001 element; Class II transposons, e.g., TN551, Tn917, Tn3871, Tn4430, Tn4556, Tn4451, Tn4452; and other transposons, e.g., Tn554, Tn3853; Tn4001, Tn3851, Tn552, Tn4002, Tn3852, Tn4201, and Tn4003 are all favorable in the context of the present invention.

### Fungal Transposable Elements

The full range of known eukaryotic transposable elements is observed in fungal genomes, including Class I and Class II transposons (for recent reviews, see, e.g., Kempken and Kuck (1998) Bioessays 20:652; Daboussi (1997) Genetica 100:253; U.S. Pat. No. 5,985,570 “Identification of and Cloning a Mobile Transposon from ASPERGILLUS” to Amutan et al., issued Nov. 16, 1999). Evidence of transposons is frequently observed in pathogenic species, and “untamed” species in general. Multiple copies of transposons frequently exist in a fungal genome, resulting in genetic instability (sometimes referred to as “genomic plasticity”) due at least in part to stimulation of genome reorganization by transposon activity.

Filamentous fungi are unusual in that they often contain multiple nuclei per cytoplasmic compartment (are coenocytic). Cells containing genetically different nuclei are designated heterkaryons, and are formed via anastamosis (fusion of hyphae). Transpositons that would lead to lethality or other detrimental effects in a mononuclear cell are often capable of surviving in a heterokaryotic cell. This provides the significant benefits of retaining mutations that would otherwise be lost, and permitting the involvement of such mutations in genome evolution. For example, the Tad LINE-like element (of N. crassa has been shown to transpose through a cytoplasmic intermediate between heterokaryon nuclei, and can introduce itself rapidly into new genomes. This is particularly useful in the application of a pool-wise recombination format.

Some fungal species can inactivate incoming transposons, e.g., through processes designated “RIP” (repeat induced point mutagenesis) and “MIP” (methylation induced premeiotically). In Neurospora crassa RIP causes C-to-T transitions in repeat sequences at a high frequency (see, e.g., Selker (1998) Proc Nat'l Acad Sci USA 95:9430; and references therein). MIP causes methylation of cytosine in DNA repeats in Ascobolis immerses (Rossignol and Faugeron (1994) Experientia 50:307). Most fungal species having transposons lack an obvious sexual cycle (or, have one that is only rarely active). In these cases RIP and MIP is not generally a problem as long as a cross is not achieved.

The following list of exemplary fungal TEs includes elements with a Class I transposition mechanism, e.g., Hideaway, MARS1, MARS2, MARS3, MARS4, MARS5, Afut1, Boty, Cft-1, CfT1, EGH24-1, Eg-R1, Foret-1, Palm, Skippy, Repa, Fosbury, Grasshopper, Maggy, MGR583, Mg-SINE, MGSR1, Nrs1, Pogo, Tad1-1; and transposons with a Class II transposition mechanism including, Ascot-1, Tascot, F2PO8, Ant1, Tan, Vader, Restless-d1, Flipper, Fcc1, Fot1, Fot2, Impala, Hop, MGR586, Pot3, Pot2, Nht1, Guest, Pce1, PSR, and Restless.

Transposable elements have likewise been isolated from yeast (Saccharomyces cerevisiae) and are favorable in the context of the present invention. Such elements include Ty1, Ty2, Ty3, as well as δ, σ, τ, and Ω elements.

### Transposable Elements in Other Eukaryotes

In addition to the previously enumerated transposable elements, numerous transposable elements have been characterized from multicellular eukaryotes, including both plants and animals. For example, numerous retrotransposons have been described in plant species. Such retrotransposons mobilize and translocate via a RNA intermediate in a reaction catalyzed by reverse transcriptase and RNase H encoded by the transposon. Examples fall into the Ty1-copia and Ty3-gypsy groups as well as into the SINE-like and LINE-like classifications. A more detailed discussion can be found in Kumar and Bennetzen (1999) Plant Retrotransposons in Annual Review of Genetics 33:479. In addition DNA transposable elements such as Ac, Tam1 and En/Spm are also found in a wide variety of plant species, and can be utilized in the present invention.

Similarly, many transposons useful in the context of the present invention have been identified in animal species. To date, active transposons have been isolated from invertebrate species, while inactive elements have been found in several vertebrate genomes. For a recent review, see, Plasterk and Izsvak (1999) Resident aliens in Trends in Genetics 15:326. In particular, transposons of the Tc1/mariner and Fot/Pogo groups can be favorably utilized in the present invention. For example, various inactive elements, from a single host species, or from several species, any number of which can be active or inactive in their respective hosts, can be recombined according to any of the recombination formats described herein, and selected for a desirable level of transposition activity in a target cell type.

### EVOLVING TRANSPOSABLE ELEMENTS WITH DESIRED PROPERTIES

Sequences derived from any of the above, or other, transposable elements can be recombined and the recombinant products evaluated for the acquisition of desired properties. Among the many properties that can be achieved by the methods of the invention are increased or decreased specificity of integration, host adaptation, increased or decreased recombinase activity, increased or decreased transposase activity, increased or decreased recombinase specificity, increased or decreased transposase specificity, desired size of the exogenous DNA transposed, copy number of integrated elements, increased or decreased efficiency of transposition, increased or decreased preference for episomal targeting, increased or decreased preference for chromosomal targeting, increased efficiency of integration into non-supercoiled DNA, and increased efficiency of in vitro transposition, etc. Numerous assays useful for detecting transposable elements and their components with these and other properties are available to one of skill in the art.

In many cases, desired outcomes can be achieved by focusing the recombination process on an individual component of the transposable element. The following series of illustrative examples demonstrates how individual components of transposable elements can be evolved to acquire a subset of pre-determined characteristics. These examples are provided to facilitate and not to limit the present invention. In general, the identification of recombinant polynucleotides with the specified qualities is dependent on the selection or screening protocol employed. Thus, a number of different desired properties can be selected or screened simultaneously from among the same library of recombinant polynucleotides. Indeed, such simultaneous evaluation for multiple properties can be advantageously employed to identify recombinant polynucleotides that are improved with respect to multiple properties when compared to the parental sequences that were the subject of the diversification reactions.

### Specificity of Integration Site

The inverted repeats flanking an IS element or transposon are recognized by the transposable element's transposase and influence the sequences into which the element will transpose. Some ISs and TNs are very specific for a particular target sequence and thus integrate into a genome relatively non-randomly, i.e., with site specificity. Others are less specific and integrate in an essentially random manner. The Inverted repeats (e.g., derived from a variety of naturally occuring or mutant transposable elements, or artificially synthesized degenerate oligonucleotides) of ISs and TNs can be recombined, e.g., shuffled, mutated or otherwise modified and screened for a change in specificity, i.e., either more specific integration or more random integration. These sequences can also be shuffled, mutated or diversified by other diversity generating method, and screened for the ability of a new IS or TN incorporating the diversified repeats to efficiently transpose in a new host. For example, a library of TNs differing in the sequences of their inverted repeats are delivered to a target cell or organism of choice. To screen for an increase in the specificity of integration, a screening method involving the detection of integration into a pre-determined sequence can be used. For example, a specific target sequence, such as green fluorescent protein (GFP), is introduced into a chromosome or episome maintained in the chosen cell. Cells losing fluorescence are enriched for those having TN integrations into the target sequence within the GFP gene. TNs having integrated into the target sequence are selectively amplified from a pool of the gDNA isolated from the non fluorescent colonies by PCR. The primers used in this reaction are hybrid sequences of the inverted repeats and the target sequence. In this manner, only TNs that have specifically inserted into the target sequence are recognized by the primers and amplified. The resulting TNs are cloned, the ends recombined, and the process performed recursively until the optimal level of specificity has been obtained.

To screen for reduced specificity of insertion, a library of inverted repeat sequences, e.g., in the context of a TN, or vector incorporating a TN, is delivered to a target cell population. Cells are then selected for insertion of the TN, for example by growing in the presence of a drug for which the TN carries a resistance gene. The cellular DNA is isolated and cleaved with a restriction enzyme outside the TN. The cleaved DNA is then size fractionated, e.g., by agarose gel electrophoresis. The more specific the target site of insertion, the smaller the variation in the size distribution of the cleaved integration products. For example, a TN with a strict requirement for a specific target sequence exhibits a single band, or a few bands corresponding to the precise number of perfect matches in the cell's DNA. In contrast, a TN with low sequence specificity for integration exhibits a broad spectrum in its size distribution, e.g., a smear. TNs from cells having insertions in a distribution of pathways are amplified by the PCR, cloned, recombined, and the process is repeated until the desired level of specificity/randomness is detected.

### Copy Number

IS/TNs range in the number of integrated copies found in each cell. While the exact determinant of copy number is unknown, it is likely that the inverted repeats influence this property. Thus, a library ISs or TNs incorporating diversified, e.g., shuffled, inverted repeats can be screened for a change in cellular copy number. A library of TN:inverted repeats (as described above) including a gene for which copy number is quantitatively detectable, e.g., kanamycin resistance, is prepared. The library is delivered to a population of cells, and the cells are selected for resistance to increasing concentrations of kanamycin. The TNs from highly resistant cells are amplified by PCR, recombined, and the process is repeated until sufficient resistance and, thus, TN copy number is obtained. Total TN copy number and distribution within the cell can be assessed by genomic southern blot analysis using the TN as a probe.

### Host Adaptation

Since most genomes contain resident ISs and TNs, there are also resident transposases. Diversification, e.g., by shuffling, of the inverted repeats can lead to inverted repeat sequences recognized by these resident transposases. This provides one approach to adapting an IS or TN to a new host cell: adapting the inverted repeats to the transposases already residing in the target cell. A library of mini-TNs, i.e., transposons lacking an encoded transposase, of differing inverted repeats containing a selectable marker is delivered to a population of cells believed to possess resident transposases. The cells are selected for integration of a TN, e.g., by selection of the incorporated marker. The total number of selected cells from the library is compared to that obtained from a population of cells receiving a control, e.g., a TN having a parental set of inverted repeats. An increase in the presence of integrated TNs indicates enhanced transposition as a result of resident transposases that recognize variant inverted repeats generated by the diversification process(es). TNs from the selected cells are amplified by PCR, recombined, and the process is repeated until the desired transposition frequency is obtained. Transposition as opposed to homologous recombination is confirmed by identification of integration sites by sequencing outward from the inserted TNs.

### Increased Efficiency of Transposition

In addition, a library of variant, e.g., shuffled inverted repeats, e.g., TNs incorporating shuffled inverted repeats can be screened for variants that are more efficiently recombined by a particular transposase, i.e., the variants can be screened for hyper-transposable elements. To identify hyper-transposable elements, cells transformed with a TN library are selected for insertions at different periods of time after transformation. Cells that obtain TN insertions at a time point that is earlier than those transformed with the wild-type TN likely transpose with greater efficiency. These hyper-transposons are amplified from the selected cells, and the process is repeated until the transposition frequency has reached a desired level.

### Transposases

Like the inverted repeats, transposases also affect the sequence specificity, the host adaptation, and the recombination efficiency of an IS or TN. Transposases can be found as single or multiple open reading frames. Many are encoded by two overlapping open reading frames such that during translation the two proteins are fused as a single polypeptide. In some cases the two open reading frames are translated both as separate proteins as well as a fusion protein. In some cases one can bind the inverted repeat sequence and inhibit the binding of the active transposase, thus, acting as a regulator, i.e., a trans-dominant regulator, of the transposase. Diversifying, e.g., by shuffling, sequences that encode transposases can be used to improve many of the same IS and TN properties as described above for the inverted repeats. Diversified transposases can be screened for recombination site specificity, i.e., more specific or more random, host adaptation, hyper-recombination, cell copy number, and the ability to mobilize other ISs and TNs within a host cell in which the transposase is expressed. Hyper-recombinogenic transposases expressed in a cell can be used to catalyze IS and TN mediated rearrangement of the cells genome, thus providing a powerful method of creating diversity within a cell population. The screens and selections described previously for site-selectivity, copy number, strain adaptability, transposition frequency, etc, can be carried out as described in the previous section.

### Targeted Insertion into a Chromosome

ISs and TNs that undergo site specific integration do so by transposase assisted recombination. Although formally considered non-homologous recombination, the process is largely directed by a limited homology between the inverted repeats and a chomosomal insertion site. Homologous recombination between such limited regions of homology is mediated by the action of the transposase. Transposases that are evolved to work with specifically designed (“designer”) inverted repeats, can be used to direct gene(s)/sequences/libraries flanked by the designer inverted repeats to specific chromosomal locations. This simple approach for targeting genes to the chromosome provides many advantages over current systems such as suicide delivery vectors. One application is to deliver fragment libraries into chromosomal expression vectors, i.e., just down stream of specific promoter or operator sequences. For example, a transposase can be evolved to target a transposon having designer inverted repeats corresponding to a specific chromosomal sequence. The resulting integration places the TN and the DNA fragments between the flanking repeats to a sequence specific locale. This process resembles gene replacement by homologous recombination rather than that typically catalyzed by a transposase. One application is the construction of a chromosomal expression cassette into which one can target any DNA, e.g., a gene of interest, to be expressed (chromosomal expression is preferred in industrial applications since it avoids the issues of plasmid loss and instability). The evolved TN/transposase system provides the tools to deliver any gene of interest to the chromosomal expression cassette such that the DNA is properly expressed. Such an approach obviates the need to carry out two steps of recombination as is required for classic gene replacement, such as that employing suicide vectors.

### Integration into Non-Supercoiled DNA

Many transposable elements, and their transposases, e.g., the TN5 transposase, as well as their hyper-recombinogenic variants, mediate integration into supercoiled DNA with much higher efficiency than they mediate integration into non-supercoiled or relaxed, e.g., linear, DNA. As purified DNA, e.g., purified genomic DNA, is typically sheared, it is not supercoiled. Thus, the efficiency of transposition mediated by such transposases, e.g., the TN5 transposase, is not optimal. To improve the efficiency with which a transposase promotes integration into non-supercoiled, i.e., relaxed, DNA, extracts of host cells, such as B. subtilis, expressing variant transposases are incubated with a mini-TN carrying a drug resistance cassette and cellular genomic DNA, under conditions suitable for transposition, e.g., in the presence of Mg²⁺. Samples of the incubation are then transformed into host cells, e.g., Bacillus host cells, and the cells are screened for resistance conferred by the drug resistance marker. Alternatively, extracts from cells expressing variants can be incubated with a transposon and a single linear fragment of “recipient” DNA. Pooled samples are separated by electrophoresis and an increase in the molecular weight of the recipient Dna due to transposon integration is detected. In either case, samples expressing transposases resulting in integration into non-supercoiled DNA are isolated, e.g., by deconvolution of the samples, and can be further improved as desired.

### In Vitro Transposition

Isolated transposases have been found to catalyze recombination between polynucleotide substrates in vitro. In particular, a variant form of TN5 has been proposed to efficiently mediate recombination between a polynucleotide having 19-bp TN5 outer end recognition sequences and a target polynucleotide (see, e.g., U.S. Pat. No. 5,965,443 “System for in vitro Transposition” to Reznikoff et al., issued Oct. 12, 1999, and U.S. Pat. No. 5,948,622 “System for in vitro Transposition” to Reznikoff et al. issued Sep. 7, 1999). The present invention can be used to evolve a wide variety of transposases that mediate transposition between DNA molecules in an acellular reaction mix. For example, acellular reaction mixes, each having a donor polynucleotide with transposase recognition sequences (e.g., inverted or end repeats), a target polynucleotide with which the donor can recombine, and a variant transposase expressed from a library of transposase encoding sequences or transposable elements are evaluated for frequency of recombination, e.g., by detecting a size difference between the donor, target, and recombined or “transposed” product by agarose gel electrophoresis. Library members can be evaluated singly or in pools.

Transposases with increased activity are useful, e.g., in the context of whole genome shuffling, as mediators of genetic change in cells. Improved transposases bind polynucleotides, e.g., having a gene of interest such as a marker, flanked by the appropriate recognition sequence. The complex, or “transposome” can be isolated, conveniently stored and handled, and subsequently introduced, e.g., by electroporation, into a cell of choice where the transposome effectively mediates genetic recombination. The result of the transposome mediated recombination is to introduce the donor polynucleotide at, e.g., essentially random, locations in the genome creating a library of insertional mutant cells with a variety of structural and regulatory alterations. Such libraries are optionally screened for desired phenotypes. One such method is proposed in PCT Application No. WO 00/17343 by Reznikoff et. al., “Method for Making Insertional Mutations,” published Mar. 30, 2000.

### Multi-Component Formats

ISs and TNs range in size from less that 1000 base pairs (ISs) to greater than 60 kb (TNs). In some cases, the properties of an individual IS or TN are not solely a property of the inverted repeat or the transposase, but rather are a holistic property of the IS or TN. Thus complete ISs and TNs can be diversified, e.g., by shuffling, and screened for any of the properties described above. For example, the size of internal DNA that can be effectively mobilized by an IS or TN is an important property with respect to its use as a vector. For the application of TN mediated whole genome shuffling, it is desirable to deliver and mobilize TNs carrying large gDNA fragments. Evolving an IS and/or TN to efficiently mobilize DNA fragments of a desired size is thus a preferred application. A fragment of DNA of desired size containing a gene for which there is a selection is cloned within a library of TNs. The library is delivered to a population of cells, and cells having insertions are selected. TNs from the selected cells are amplified by the PCR. The amplified population is separated by agarose gel electrophoresis and those having a molecular weight corresponding to a TN maintaining the complete inserted DNA are isolated, recombined, and reevaluated. This process is repeated until a TN capable of stably carrying DNA of the desired size is obtained.

### DIRECTED EVOLUTION OF TRANSPOSABLE ELEMENTS

A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or directed evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids with or which confer desirable properties, or that encode proteins with or which confer desirable properties. Following diversification by one or more of the methods herein, or otherwise available to one of skill, any nucleic acids that are produced can be selected for a desired activity or property, e.g. transposable elements with improved in vivo or in vitro transposition efficiency, integration specificity, copy number, host specificity, etc. This can include identifying any activity that can be detected, for example, in an automated or automatable format, by any of the assays in the art, e.g., as described above. A variety of related (or even unrelated) properties can be evaluated, in serial or in parallel, at the discretion of the practitioner.

Descriptions of a variety of diversity generating procedures for producing modified transposable element nucleic acid sequences are found in the following publications and the references cited therein: Soong, N. et al. (2000) “Molecular breeding of viruses” Nat Genet 25(4):436-439; Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly:In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the asubunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986) “Oligonucleotide-directed double-strand break repair in plasmids of Escherichia coli: a method for site-specific mutagenesis” Proc. Natl. Acad. Sci. USA, 83:7177-7181; and Arnold (1993) “Protein engineering for unusual environments” Current Opinion in Biotechnology 4:450-455). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications and applications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” WO 01/23401 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter.

In brief, several different general classes of sequence modification methods, such as mutation, recombination, etc. are applicable to the generation of transposable elements (e.g., transposons, insertion sequences, and their components) with desired properties, and set forth, e.g., in the references above.

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751. Thus, transposable elements with desired properties, such as increased transposase activity, increased in vitro transposition activity, altered host specificity, targeted insertion, and the like, can be produced by in vitro recombination procedures.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. Thus, in vivo recombination procedures can be employed to recombine and select transposable elements with improved properties.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., WO 00/04190 by del Cardayre et al., also entitled “Evolution of Whole Cells and Organisms by Recursive Sequence Recombination.” Such methods can be used to generate variant transposable elements with new and improved characteristics, e.g., by recombining genomes harboring one or more transposable element, and, optionally by introducing into such cells, additional sequences derived from libraries of nucleic acids, e.g., comprising components of one or more transposable element.

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561 by Crameri et al., “Olgonucleotide Mediated Nucleic Acid Recombination;” WO 01/23401 by Welch et al., “Use of Codon-Varied Oligonucleotide Synthesis for Synthetic Shuffling;” WO 00/42560 by Selifonov et al., “Methods for Making Character Strings, Polynucleotides and Polypeptides Having Desired Characteristics;” and WO 00/42559 by Selifonov and Stemmer “Methods of Populating Data Structures for Use in Evolutionary Simulations.”

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/ gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 by Selifonov et al., “Methods for Making Character Strings, Polynucleotides and Polypeptides Having Desired Characteristics” and WO 00/42559 by Selifonov and Stemmer “Methods of Populating Data Structures for Use in Evolutionary Simulations.” Extensive details regarding in silico recombination methods are found in these applications. This methodology is generally applicable to the present invention in providing for recombination of transposable elements and their components in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nucleasebase removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity into transposable elements and their components. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “Methods for Generating and Screening Novel Metabolic Pathways,” and Thompson, et al. (1998) U.S. Pat. No. 5,824,485 Methods for Generating and Screening Novel Metabolic Pathways) and their use to identify protein activities of interest has been proposed (In addition to the references noted above, see, Short (1999) U.S. Pat. No. 5,958,672 “Protein Activity Screening of Clones Having DNA from Uncultivated Microorganisms”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/ or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “Production of Enzymes Having Desired Activities by Mutagenesis.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

Any of these or other available diversity generating methods can be combined, in any combination selected by the user, to produce nucleic acid diversity, which can be screened or selected for using any available screening or selection method to identify evolved transposable elements or TE components as described herein.

In one aspect, the present invention provides for the recursive use of any of the diversity generation methods noted above, in any combination, to evolve nucleic acids or libraries of recombinant nucleic acids that encode enzymes involved in transposition or that are transposable elements, including both cis- and trans-acting mobilization functions. In particular, as noted, the relevant nucleic acids, e.g., TNs, Iss, transposase, inverted repeats, etc., can be modified before selection, or can be selected and then recombined, or both. This process can be reiteratively repeated until a desired property in obtained.

Regardless of the diversity generating method or methods employed, identification of novel transposable elements and TE components involves one or more screening and/or selection protocol distinguishing nucleic acids encoding products with desired properties. In some instances, the desired property or characteristic relates to the nucleic acid, e.g., hybridization, amplification, or the like. However, in many cases the desired characteristic relates to a functional property conferred by the recombinant nucleic acid, e.g., inverted repeat, ORF encoding a transposase, etc, expressed in situ.

### TRANSPOSABLE ELEMENTS AS VECTORS

The breeding of a population of microbes can be facilitated by the use of “mobilizable” genomic libraries that are delivered via transposable elements. In general, genomic DNA from a population of organisms is fragmented and cloned within a transposable element. This “transposable library” is then delivered to a desired host or a population of hosts, such as the original population of organisms. Delivery can be via transformation of the library on a suicide or conditionally replicative vector, e.g., by electroporation or other well-known transformation technique, or via conjugative delivery, if the library is cloned within a conjugative transposon.

There are many variations on the nature of the transposable element into which the gDNA is cloned that can alter the effectiveness of the approach. For example, the transposable element can be an insertion element, a transposon, or a conjugative transposon. These elements can be “mini-transposable elements,” such that the transposition genes are removed and provided in trans. Mini-transposable elements are preferable in some cases since incorporation into the host genome is stable in the absence of transposition factors, e.g., a transposase. Once a transposon shuffled library of microorganisms has been generated, it can be screened for desired phenotypes. The subpopulation resulting from the screening can then be further bred and screened using the same methodology until a desired phenotype is achieved.

One classic method of microbial strain improvement is expression cloning. This process involves cloning genomic DNA into an expression vector, and then transforming the expression library into a desired host organism. The transformants having improved properties are then identified by an appropriate screen or selection. A similar approach is accomplished using transposons. A genomic DNA library is cloned, e.g., into a transposon or mini-transposon and delivered to the chromosome of a target organism. In addition to delivering the library sequences, the transposable element delivery vehicle explores multiple insertion sites within the genome providing an additional empirical parameter than can be optimized in seeking the desired cell phenotype.

Transformants that have improved properties are then isolated. Since the sequence of the TN is known, PCR primers directed to the TN are sufficient to amplify the transposed gDNA. In one approach, each amplified gDNA is shuffled independently, and subcloned into the original TN delivery vector. The result is several libraries each originating from the gDNA amplified from a single improved clone. These are pooled and used to transform the original host strain, with further improvements being obtained by screening.

### GENERAL DELIVERY VECTORS

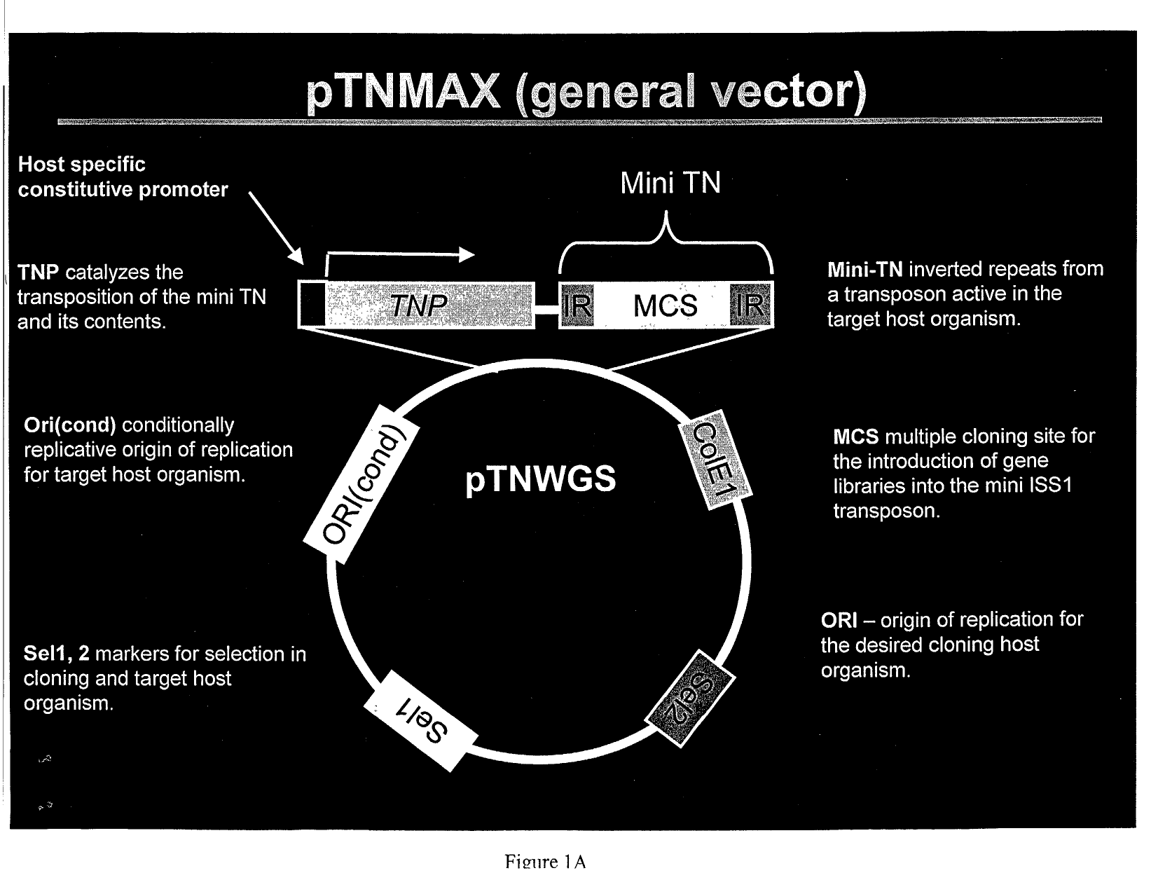

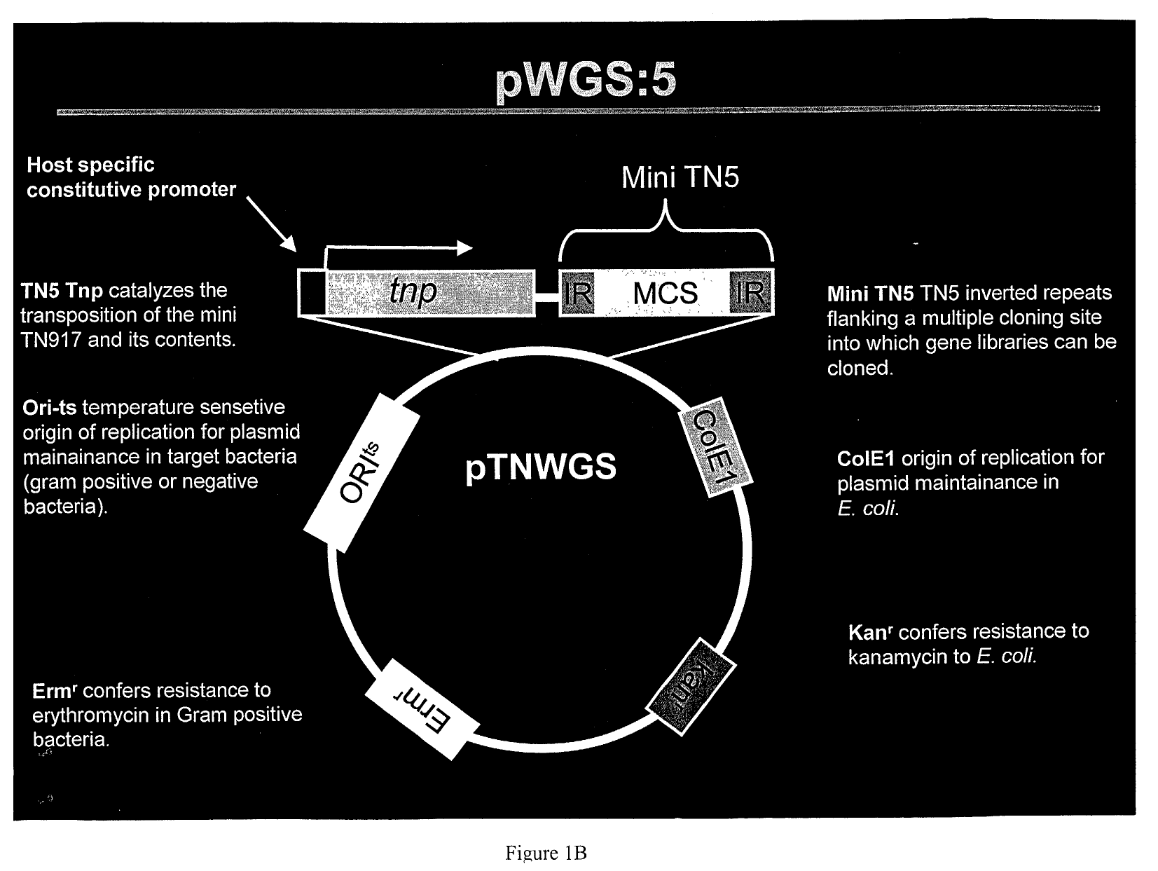

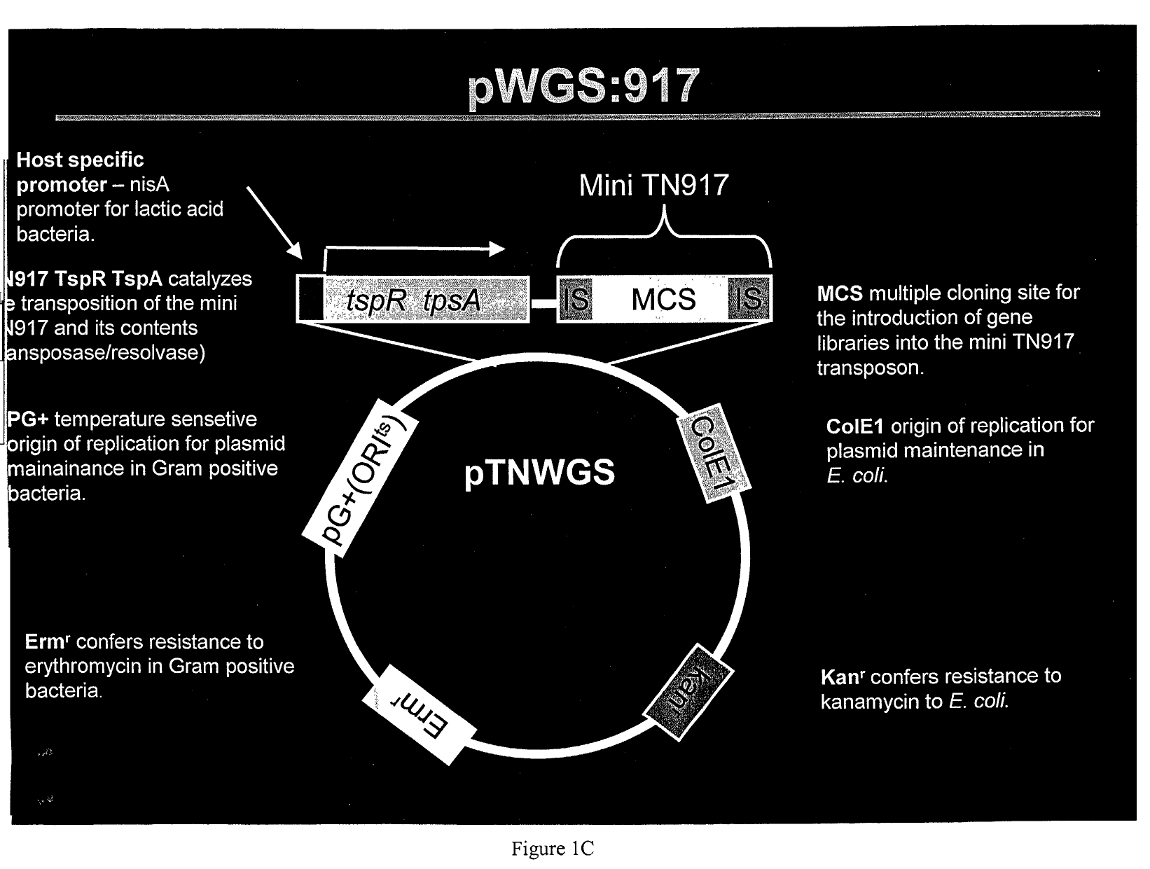

One goal for TN and IS mediated genome diversification, e.g., shuffling, is the delivery of libraries of DNA fragments to a population of cells such that that members of the library are stably incorporated into the genomes of the cells. A general set of delivery vectors are described that can be used for this purpose, see, FIGS. 1A-C. The vectors share several common components (FIG. 1A): an origin of replication active in a convenient cloning host, a conditional origin of replication for the target cell into which the library is being delivered, markers for positive selection in both hosts, a mini-transposon (two inverted repeats surrounding a multiple-cloning site), and, optionally, a transposase that catalyzes the mobilization of the sequence contained between the inverted repeats linked to a promoter that drives the expression of the transposase in the target cell. In some alternatives, the transposase is supplied in trans on a second vector or integrated into the genome of the target cell. The vectors are preferably designed in modular fashion to facilitate adaptation to new host cells or for different applications (examples are provided in FIGS. 1B and 1C). It will be appreciated that the specific choices of components are not essential to the invention and that numerous sequences are available to fulfill each function recited above. The specific choices will be apparent to those of skill in the art based on the specific application under consideration. The following examples are provided as illustration not as limitation.

### Origin of Replication for Cloning Host

Origins of replication can be derived from any plasmid that replicates in a desirable host useful for molecular cloning for the project of interest. These most often will be for E.coli, but can also be chosen for use in other common organisms such as bacillus, synechosystis, streptomyces, cornybacterium, lactic acid bacteria, yeast, and fungi. Some examples are: ColE1 series, pACYC series (p15A), RK4, pCM595, pSa, RK6, pUB110, pE194, pG+, SLP1, pMEA100, pSAM2, pSG1, pIJ408, pIJ110, pSE101, pSE211, pAMβ1, pIP501, pAC1, pRI405, pIP612, pIP613, pIP646, pIP920, pMV103, pMV141, pSF9400, p43, pSM19035, pERL1, pSM10419, pT181, pC221, pC223, pS194, pUB112, pCW7, pHD2, pC194, pUB110, pOX6, pLS11, pTA1060, pBAA1, pBS2, pUG1, pFTB14, pBC16, pBC1, pCB101, pLP1, pIJ101, pC30i1, pTD1, pKYM, φX174, pLAB1000, pWGB32, pVA380-1, pRF1, pE194, pMV158, pWV01, pSH71, pFX2, pLB4, pA1, pADB201, pKMK1, pHPK255, pSN2, pE12, pE5, pT48, pTCS1, pNE131, pIM13, pTKX14, 2 micron circle based plasmids, artificial chromosomes, etc.

### Conditional Origins of Replication

pSA3, pE194tm, pG+tm, are all temperature sensitive replicons for Gram-positive bacteria. There are also mutants of plasmid replication origins for Gram-negative bacteria that deem those plasmids conditionally replicative. Alternatively, conditional origins suitable for maintaining episomal replication in eukaryotic hosts can be employed.

### Selection Markers

Markers conferring resistance to antibiotics, prototrophy to auxotrophic organisms, or resistance to toxic compounds. Some examples are: kanamycin resistance (aph3A, and others), ampicillin resistance, macrolide-lincosarnine-streptogramin (MLS) resistance, as well as resistance to apramycin, spiramycin, hygromycin, chloramphenicol, tetracycline, and many other compounds.

### Mini-Transposon

In the context of a vector, a mini-transposon (or mini-IS) is simply the inverted repeats of a transposon or IS element flanking a sequence of DNA, most frequently a multiple-cloning site, into which a library of DNA fragments can be cloned. The inverted repeats of the transposable element used should be such that the expressed transposase on the same plasmid (or supplied in trans) recognizes them as recombination substrates. The inverted repeats and mobilization genes can originate from any TN or IS element that can function in the target host into which the mini-TN is to integrate. A partial list of possible TNs and IS elements functioning in a variety of target organisms is provided above.

### Transposase

Mobilization enzymes, i.e., transposases, are, in general, one or more enzymes, including integrases, recombinase, e.g., xis, int encoded polypeptides, that catalyze the excision and integration of the mini-TN into the target host cell genome. These genes encode enzymes that recognize the inverted repeats of the mini-transposon of the vector. These can be wild-type mobilization enzymes or ones which have been optimized by directed evolution, e.g., DNA shuffling. In many circumstances, it is most convenient to supply the transposase on the same vector as the mini-transposon, thus, in fact, supplying a transposon. In such cases, it is often preferable to locate the transposase in close proximity to the ends of the inverted repeats. The precise meaning of “close proximity” will vary from vector to vector, and can be interpreted to mean close enough to insure efficient mobilization of the mini-TN by the transposase. The requirements of the particular vector will be readily determined experimentally. In some cases this will be adjacent to one of the inverted repeats, while in other cases more relaxed requirements will be observed.

### Promoter

A promoter can be any sequence of DNA that directs the constitutive or controlled expression of the down stream mobilization gene(s), e.g., transposase, int gene, xis gene, etc. These sequences, like the conditional origin of replication are often host specific, and thus are selected to function in the host into which the mini-transposon of the vector is targeted for integration. Under some circumstances, it is preferable to use an inducible promoter that can be tightly regulated by the practitioner. In other cases, constitutive or transient promoters are selected. In some cases, the promoter is selected from among the endogenous promoters of the host cell.

### ACTIVATING DORMANT/LATENT TRANSPOSITION

Evolved mobilization enzymes (e.g., transposases, integrases, recombinases, etc.) of the present invention can be used to activate dormant transposition activities in prokaryotic or eukaryotic cells. For example, a cell population (comprising known or unknown transposable elements) can be transformed with a library of plasmids expressing, e.g., evolved mobilization enzymes of the present invention, preferably under the control of an inducible promoter, and the cell population screened for increased transposition frequency. The increased transposition frequency can be assessed relative to background (e.g., uninduced) transposition frequency by comparing the transposition frequency of a cell population transformed with plasmid expressing transposase to that of a cell population transformed with plasmid lacking transposase (or, if transposase is under the control of an inducible promoter, cells grown in the absence of inducer). For example, transposition frequency can be assessed by the generation of auxotrophic mutations in a cell population by comparing the number of cell colonies present in serial dilutions plated onto minimal media plates vs. rich media plates. Transposition frequency can also be assessed in cells by monitoring the appearance of knockout mutations in a marker gene (e.g., by loss of fluorescence when the marker gene is GFP) and/or by the appearance of papillated colonies or other morphological changes. The transposable elements (e.g., IS elements) activated by the transposase can be identified by PCR-amplifying and sequencing the knocked-out selectable marker genes.

Cells comprising dormant transposable elements identified as described above are useful in developing mutator-like strains in which transposition is activated in a controlled manner, e.g., by addition (or induction) of the cognate transposase. Such inducible mutator strains are useful for in vivo mutagenesis applications, such as evolving cells for improved phenotypes as described herein.

### TRANSPOSITION VIA INTERMEDIATE HOST

One difficulty presented by many transposable elements is the preference of the transposase for supercoiled DNA. In the absence of a transposable element vector/transposase specific for relaxed (non-supercoiled) DNA, genome diversification can be accomplished using an intermediate host organism. In the following illustrative example, transposon mediated recombination of Bacillus genomic DNA is accomplished using E. coli as an intermediate host. For example, to recombine genomic DNA between B. subtilis and another organism, genomic DNA (gDNA) from the two organisms is prepared (by standard methods). A Bacillus gDNA library is then prepared in an appropriate E. coli vector, such as a bacterial artificial chromosome (BAC) or other low copy number plasmid, e.g., pACYC, that can harbor DNA fragments of at least 2 kb (preferably greater than about 10 kb). A gDNA library of the other organism(s) is prepared in a mini-TN, such as the mini-TN5 of pMOD (Epicentre). The TN gDNA library is then integrated into the plasmid (BAC) gDNA library of B. subtilis, which is supercoiled as purified from E. coli. The TN library inserts throughout the plasmid gDNA library, resulting in a plasmid encoded TN-mediated recombinant genomic library. The products of this reaction are then transformed into E. coli to “clean up the reaction,” i.e., to fill in and ligate the broken ends resulting from the insertion reaction, and screened (or selected) for the presence of the plasmid library. Plasmid DNA is then isolated from the pool of transformants harboring the selected colonies. This isolated plasmid library is the transformed into naturally competent Bacillus, and the Bacillus gDNA is incorporated into the Bacillus genome by homologous recombination, carrying with it any genomic DNA from the donor species that has been integrated via the transposable element vector. The transformed cells are then screened or selected for cells having desired properties, such as acid tolerance, heat tolerance, or improved production of a desired metabolite, etc.

### IMPROVED VECTORS FOR INTEGRATION INTO MAMMALIAN CELLS

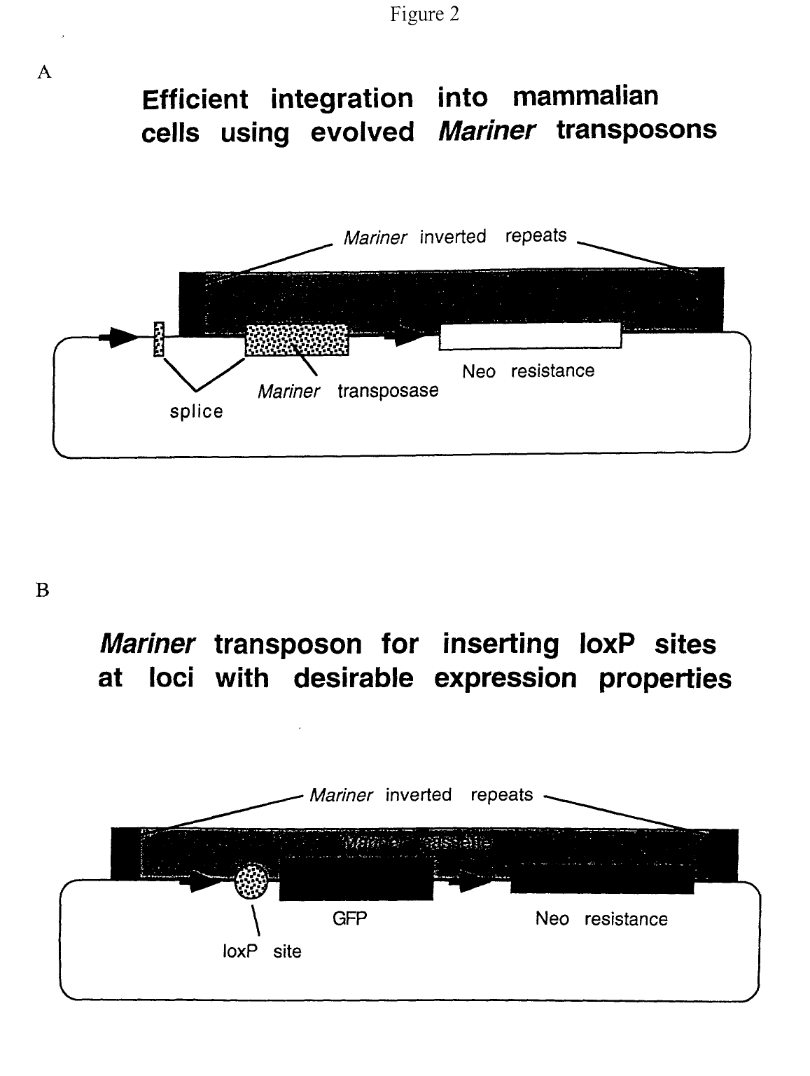

Although active transposable elements are recognized in many invertebrates, and inactive remnants of transposable elements are observed in vertebrate, including mammalian cells, no naturally transposing elements are known in mammalian cells. This limits the application of this valuable tool to mammalian cells. The present invention is used to develop transposable element vectors that efficiently integrate into mammalian, including human cells. While many sequences are suitable as substrates in the generation of such a vector, one particularly attractive candidate group of sequences are the Mariner transposable elements. Many suchTEs are known that transpose in a broad host range, including higher eukaryotic cells. To facilitate screening of a diversified library of transposable elements for their ability to mediate integration into the genome of mammalian cells a vector incorporating from 5′ to 3′: a promoter; a splice donor site; a first inverted repeat; a transposase having a splice acceptor site at its upstream terminus; a selectable marker; and a second inverted repeat. An exemplary vector is illustrated in FIG. 2A. The target cell population is transfected with the vector which transiently expresses the transposase from a message spliced between the splice donor and acceptor sites. When a transposase capable of mediating integration in the selected cell type is expressed, transposition of the sequences flanked by the inverted repeats into the cellular genome can occur. Cells that have integrated these sequences survive selection based on the selectable marker, e.g., neomycin resistance. Following integration the transposase is inactive due to a separation between the promoter and the coding sequence. The coding sequences can nonetheless be recovered by PCR and further recombined and selected, following reconstruction of the vector, if desired. The entire process can be performed recursively until a desired level of transposition is achieved.

### TRANSPOSONS AS AGENTS OF GENOME DIVERSIFICATION

Directed evolution of whole genomes, e.g., genome shuffling, is a combination of two processes: genome diversification (e.g., intra-genome shuffling) and genome recombination (e.g., inter-genome shuffling). Transposable elements affect both of these processes, and are employed in the present invention to accelerate whole cell evolution. Insertion sequences and transposons catalyze the structural and functional diversification of genomes by a variety of genetic phenomena. These include gene activation, inactivation, and attenuation, sequence inversion, duplication, deletion, and mobilization, homologous recombination, and other rearrangements. In nature, these events occur spontaneously and can also be induced by cellular stress, such as starvation or exposure to extreme environments. In addition, such events can be induced artificially by activating the enzymatic machinery of transposition, e.g., through activation of an inducible promoter.

IS elements, mini-IS elements, transposons, and mini-transposons are introduced into host cells using appropriate delivery vectors and transformation techniques. For example, plasmid vectors incorporating transposable elements can be introduced into the selected host cell population by any of a number of known techniques, e.g., microinjection, electroporation, agrobacterium mediated transformation, calcium phosphate precipitation, etc. Alternatively, isolated transposomes can be introduced, e.g., by electroporation, into the cells. Which technique is selected is largely a matter to be determined by the particular application and host cell type, and will be apparent to one of skill in the art.

Integration and mobilization of these elements within the genomes of the transfected cells result in the diversification of the cell population by the mechanisms described above. This diversification can be iteratively induced by either transiently expressing the transposase or by exposing the population to periodic stress. For example, an IS element known to be induced by nitrogen starvation is delivered to a population of cells on a plasmid. The cell population is then grown under nitrogen limiting conditions to induce the intra-genomic transposition of the IS element throughout the genomes of the transfected cells. The result is a diverse population of cells having different chromosomal insertions and rearrangements. An alternative is to deliver a mini-IS element, in which the transposase has been removed from within the mobile element and placed elsewhere in the genome under an inducible promoter. Upon induction, the transposase is expressed and catalyzes the mobilization of the mini-IS elements and the corresponding genomic rearrangements. The difference between these two strategies is that the mini-IS elements cannot mobilize without the transposase being induced or provided in trans. Thus, the final strains will be more stable than those having naturally inducible transposases within the IS elements. Processes using natural IS elements or transposons access the natural mechanisms of genome plasticity, while those using the mini-IS elements and transposons are designed to accelerate and control these natural processes. Both are of value for the purpose of directed cellular evolution.

The population resulting from the IS element mediated diversification is enriched for improved variants by either screening or selection. One preferred method for the enrichment of organisms having improved environmental tolerance is to grow the population under increasingly stringent conditions in a chemostat or turbidostat. The growing populations are slowly exposed to conditions of increasing stringency, such as increased temperature or pH. Variants having improved tolerance overtake the population. It is important that conditions are not made so stringent that no cells survive or that only a single clone survives. Rather, genetic diversity within the tolerant population is maintained and selective conditions are generally such that a group of improved variants survive. This tolerant population can then be further diversified as a result of the stressful conditions naturally inducing the mobilization of the IS elements i.e., continuously adapting to the conditions imposed. Alternatively, the population can be diversified by transiently inducing the expression of a transposase after each step of increased environmental stringency. An additional strategy of enrichment is the oscillation between stringent and permissive conditions. The diverse population is gradually exposed to an environmental challenge such that a significant portion of the population is removed. The survivors are gradually returned to permissive temperature, where they further diversify (naturally or by induction), and then gradually back to conditions slightly more stringent than the previous challenge. This process is repeated recursively until the population can tolerate no further increase in challenge. At this point, the evolutionary process benefits from the recombination of genetic information between cells existing within the population, e.g., by cellular fusion, or other described methods.

The genetic information within a population of improved cells can be recombined by any of the previously described methods for whole genome recombination, e.g., shuffling. Whole genome recombination of the improved population will generate a combinatorial genetic library of cells and/or genomes having all possible combinations of the genetic rearrangements present in the improved population. Further details regarding whole genome shuffling are provided, e.g., in USSN 116,188 and PCT publication WO 00/04190 (Jan. 27, 2000) “Evolution of Whole Cells and Organisms by Recursive Sequence Recombination,” by del Cardyre et al. filed Jul. 15, 1999. This library is then subjected to further phenotypic enrichments and intra-genomic shuffling. The iterative process of intra-genomic shuffling enrichment, and inter-genomic shuffling is cycled until the phenotype of interest is achieved.

### TRANSPOSOME MEDIATED GENOME DIVERSIFICATION

Diversification of whole genomes can also be accomplished in vitro using transposomes to mediate the recombination events. This method provides a means of efficiently recombining the genomic DNA from multiple different organisms in vitro. Large fragments of genomic DNA are recombined, e.g., shuffled, in vitro by transposasemediated non-homologous recombination. The resulting diverse library is then delivered to a target host organism, e.g., where homologous recombination of the library with the host genome results in chromosomal variations that mimic in vivo transposition of heterologous DNA.

Genomic DNA is purified using standard procedures from various sources according to the properties and diversity desired. Typically, genomic DNA from organisms expressing a desired phenotype or expressing a phenotype related to the desired phenotype is utilized. Examples of such sources of genomic DNA are: genomic DNA of different species or strains of microorganisms, such as Yeast, E.coli, Pseudomonads, Bacillus; genomic DNA from cultured organisms originating in environments likely to encode a desired property or phenotype; genomic DNA from mixed microbial cultures or from uncultured environmental samples; genomic DNA from diversity created in the laboratory through NTG, UV mutagenesis or adaptation to certain selective conditions; and cDNA libraries of various organisms, species and strains, e.g., as indicated above, etc.

In one embodiment, the “donor DNA” and the “acceptor DNA” are pools of genomic DNA originating from the same diverse population of organisms. For example, genomic DNA from several organisms to be recombined, e.g., shuffled, is isolated. This DNA is pooled and then divided. One portion is used to construct a transposome library, the “donor DNA,” while another portion is used as “acceptor DNA.” In vitro transposition of the donor and acceptor pools results in the breeding of the two populations creating a combinatorial genomic library.

The source DNA is fragmented, e.g., with suitable restriction enzymes, to yield a random collection of clonable DNA fragments. These fragments are cloned between insertion sequence (IS) elements such that the genomic DNA fragments are flanked by IS elements, which under suitable conditions can transpose randomly into DNA. For example, the genomic fragments are cloned into a mini-transposon (e.g., Tn5, a shuffled mini-transposon) which contains recognition sequences (e.g., the 19-bp Tn5 transposase Mosaic End (ME) recognition sequences, inverted repeats recognized by a shuffled transposase).

The cloned library is mixed with the corresponding transposase, which binds to the recognition sequences and forms a stable complex, or transposome. Under appropriate storage conditions, e.g., Tn5 based transposomes are stable in the absence of Mg++ ions, the transposomes are stable, and can be purified and/or stored until added to a reaction mix. Genomic recombination is achieved by mixing the transposomes incorporating the donor DNA with acceptor DNA, e.g., from one or more target organisms under conditions favorable for recombination. Conditions favorable to the activity of a particular native or recombinant, e.g., shuffled, transposase can vary, and such conditions can be determined empirically to optimize recombinatorial activity of a particular transposome complex. Transposition results in the random insertion of the “mini TN library” into the acceptor DNA. The result is a library of acceptor DNA harboring integrated fragments of heterologous DNA.

In some instances, it is desirable to bias the in vitro transposition reaction with one or more nucleic acid of interest in order to create further diversity in the genomic library. This can be accomplished by spiking the reaction with transposomes including the nucleic acid of interest, such as a desired promotor, regulatory elements, e.g., terminator sequences, antiterminator sequences, Start codons, Stop codons, etc., libraries of shuffled genes, selected genes, or IS elements.

Additional diversity is introduced by performing the above process recursively. For example, a pool of recombinant nucleic acids resulting from a first in vitro transposition reaction is divided, and one portion is digested, and cloned into a mini-transposon as described above. Transposomes incorporating this new library are then prepared and used to mediate transposition, e.g., in a second portion of the recombinant nucleic acids or genomic DNA from one or more parental species or strain. This process can be carried out for as many cycles as is desired to generate the appropriate level of diversity.

Optionally, the recombined nucleic acids are digested with suitable restriction enzymes to various sizes to facilitate their uptake and integration into host cells. These linearized fragments, or the undigested library are then delivered into suitable host cells by a variety of methods, depending on the host cell selected. For example, many microorganisms, e.g., Bacillus Subtilis, Acinetobacter sp., Synechocystis sp., Streptococcus sp., etc. have natural competence mechanisms that mediate uptake of DNA molecules with high efficiency. Alternatively, the recombinant nucleic acids can be cloned into suicide vectors and introduced through standard transformation techniques such as electroporation. Suitable recipients for this approach include E.coli, Saccharomyces sp., Streptomyces sp., etc. Yet another alternative is the direct transformation, e.g., by electroporation of the recombinant nucleic acids into such host cells as yeast and other eukaryotic cells including mammalian host cells. In still another alternative, the recombinant nucleic acids are packaged into and delivered by various bacteriophages known in the art.

Following introduction of the recombinant nucleic acids into a population of host cells by any of these various means, a portion of the delivered DNA recombines with the host genome, generally by homologous recombination. This recombination results in “gene replacement” of the host DNA with the recombinant nucleic acids generated by the in vitro transposition reaction, e.g., having inserted additional material by the in vitro integration of the donor DNA. The resulting cell population is then screened or selected for variants having evolved toward a desired phenotype. This population is then, optionally, recombined either with itself or with other donor or acceptor DNA, and the process is repeated until the desired phenotype is achieved.

### GENE IDENTIFICATION USING TRANSPOSABLE ELEMENTS

IS elements and transposons are common tools for introducing mutation in cells. These mobile genetic elements are delivered to cells using an appropriate delivery vector, tranposition is selected for and the resulting insertion mutants are screened for a phenotype of interest. Affected loci can be mapped by sequencing out from the TN into the chromosome to identify the chromosomal location. This process can be used to identify genes to be evolved, e.g., shuffled, for the improvement of desired phenotypes.

A TN harboring a drug resistance marker and origin of replication for an appropriate host organism is used to mutagenize a target organism, for example lactobacillus. The insertion mutants are screened for a desired phenotype, such as the ability to grow at low pH. Genomic DNA from tolerant cells is isolated and digested with a restriction enzyme not located within the TN. The digested DNA is diluted, circularized by ligation, and used to transform cells than can propagate the circularized DNA using the origin within the TN. The cloned gDNA is then sequenced to identify the affected loci. The encoded genes can then be diversified by any of the directed evolution technologies, e.g., including MolecularBreeding™, described herein, expressed in the original organism and screened for further phenotypic improvements. Alternatively, the cloned gDNA need not be sequenced, but rather can be evolved, e.g., shuffled, blindly using known sequences within the TN to tag sequences for amplification and recovery.

One such application is the identification of genomic loci that engender a desired level of gene expression. One difficulty encountered in efforts to produce improved phenotypes, is that even after optimizing a given gene contributing to the desired phenotype, significant variation can result after integration as a transgene. This is often due to differences in expression level of the optimized gene. The present invention provides vectors and methods for identifying genomic loci that result in the desired level of expression of a transgene integrated therein. For example, a target cell is co-transfected with a transiently replicating vector bearing inverted repeats, e.g., from a transposable element such as Mariner, a loxP site, a visible marker such as GFP and a selectable marker such as neomycin resistance. An exemplary vector is illustrated in FIG. 2B. The transfected cells are exposed to neomycin and resistant cells are selected. These transfectants are then evaluated for a desired level of gene expression, e.g., GFP expression. Subsequently, a gene of interest, such as a gene optimized by shuffling, mutation or other diversity generation methods, can be integrated into the chromosomal locus by recombination at the loxP site mediated by a Cre recombinase.

### GENETIC BARCODES

A further utility of using TNs, or mini-TNs, is to create tagged mutants that can be described as a composition of matter. The location of a TN within a genome of a target organism can be determined by known method, e.g., sequencing of flanking regions as described above. The TN used to create the strain can contain a predesigned sequence of DNA, a DNA barcode, that identifies theTN and the strain to have been created by a particular producer or manufacturer. A simple PCR reaction from the strain will amplify the sequence which can then be diagnostically sequenced to confirm its origin.

### INCREASED ORGANIC ACID TOLERANCE IN LACTOBACILLLI

In the fermentation and bioprocess industries the optimal conditions for the organism and those for process economics do not necessarily coincide. This often poses problems of combining different phenotypes observed in various hosts into a single ideal production host, the goal being to evolve a production host that functions under the desired conditions. In-spite-of our significant knowledge in correlating genotypes with phenotypes in well known organisms like E.coli, Yeast, and Bacillus, it is extremely difficult to integrate multiple phenotypes into a single host using present day tools of molecular biology, classical mutagenesis, and/or metabolic engineering.

For example, a lactobacillus strain able to tolerate the low pH, and high concentration of organic acid required to produce high yields of lactic acid is of significant economic value. The described invention provides a method for generating such an organism. A population of lactobacilli each having traits desired for the industrial fermentation of lactic acid, e.g., heat tolerance, high volumetric yield, high lactic acid titer, etc., are grown and their genomic DNA (gDNA) is isolated and pooled. The gDNA is then fragmented, e.g., by limited digestion with a desired four base cutting restriction endonuclease. Fragments, typically of greater than 10 kb, are isolated and cloned within a “mini TN or IS” located on an appropriate plasmid, e.g., pTNWGS:TN5 (FIG. 1B). To facilitate this cloning step, a multiple-cloning site (MCS) is positioned between the two end repeat sequences of TN5. This miniTN is flanked by the transposase gene(s) of TN5 that will catalyze, in trans, the excision and integration of the mini-TN and its contents. The plasmid pTNWGS:TN5 also contains the ColE1 origin of replication, a gene conferring positive selection in E. coli (such as ampicillin resistance, kanamycin resistance, chloramphenicol resistance, etc.) and in Lactobacilli (such as erythromycin resistance, kanamycin resistance, chloramphenicol resistance, tetracycline resistance, etc.), and a thermosensitive replicon functional in Lacotbacillus such as pG+.

The pTNWGS library ligation is transformed into E.coli (preferably deficient in restriction and modification systems). Transformants are pooled and the plasmid DNA is isolated. The pTNWGS library is then transformed back into one or all of the starting Lactobacilli strains. Transformants are selected, transferred to the nonpermissive temperature for pG+ and incubated to select for the loss of pTNWGS and the integration of the miniIS library into the chromosome.

The cells are then returned to the permissive temperature, and enriched for those cells having increased tolerance to low pH in the presence of organic acids. This is achieved by inoculating a turbidostat culture and continuously challenging the growing cells with medium of lower pH and increased concentrations of organic acids.

The surviving culture is separated into individual clones by plating on solid medium, and individual colonies are picked and assessed for their ability to produce high levels of lactic acid in fresh or conditioned medium. Those clones producing high levels of lactic acid are pooled, recombined (e.g., shuffled) and screened by repeating the preceding procedure. A similar protocol is employed to produce organisms that have improved performance under a variety of extreme conditions desirable for accelerated production processes, e.g., elevated temperature, high cell-density, slow growth, high end product concentration, presence of growth inhibitors or toxins, etc.

### Serial Fermentation for Selection of Improved Industrial Phenotypes

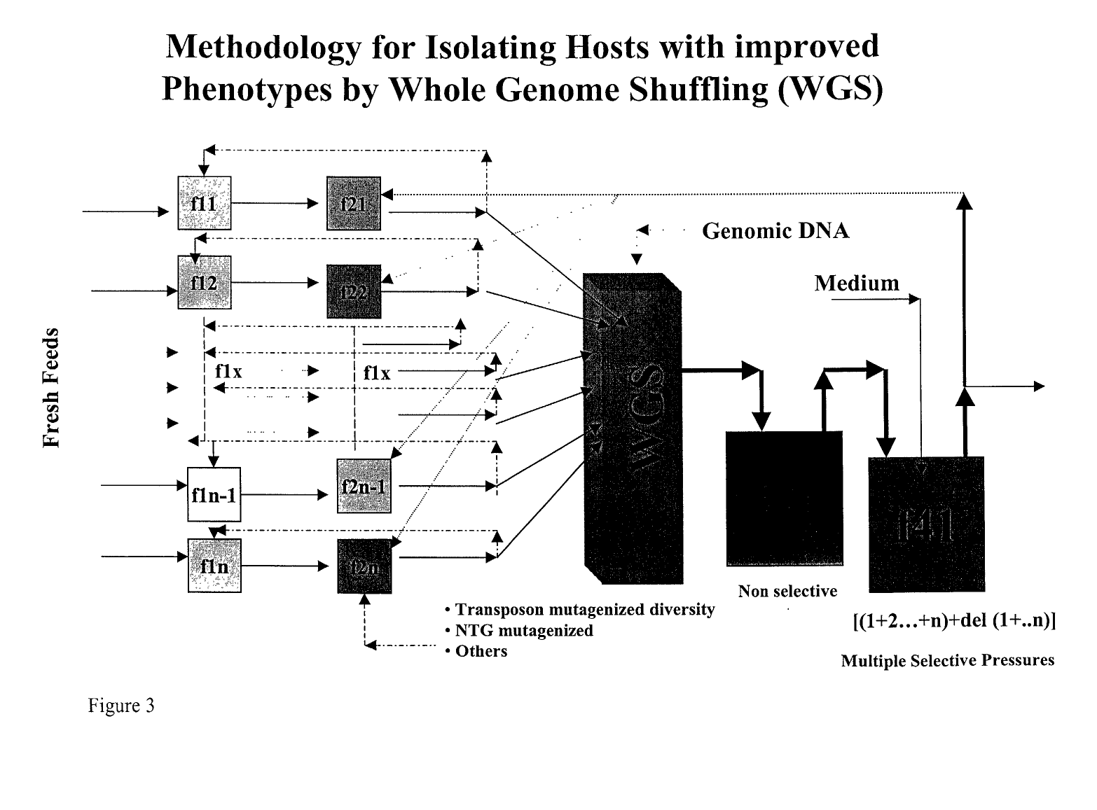

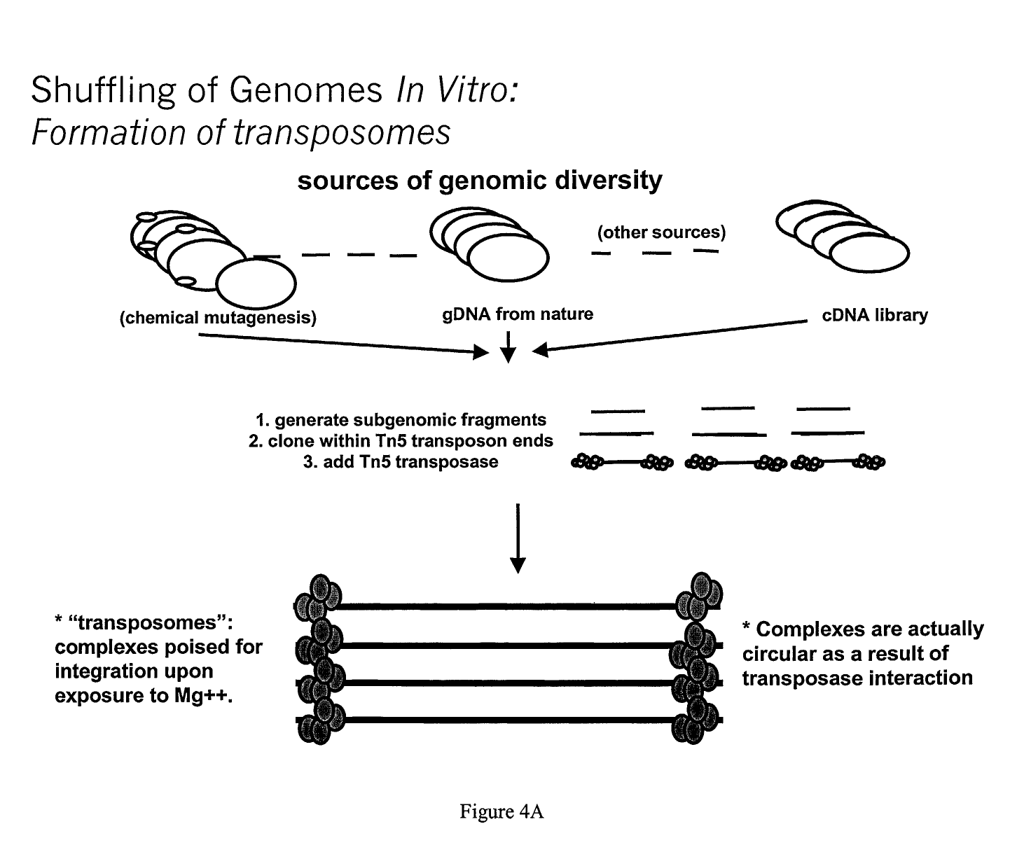

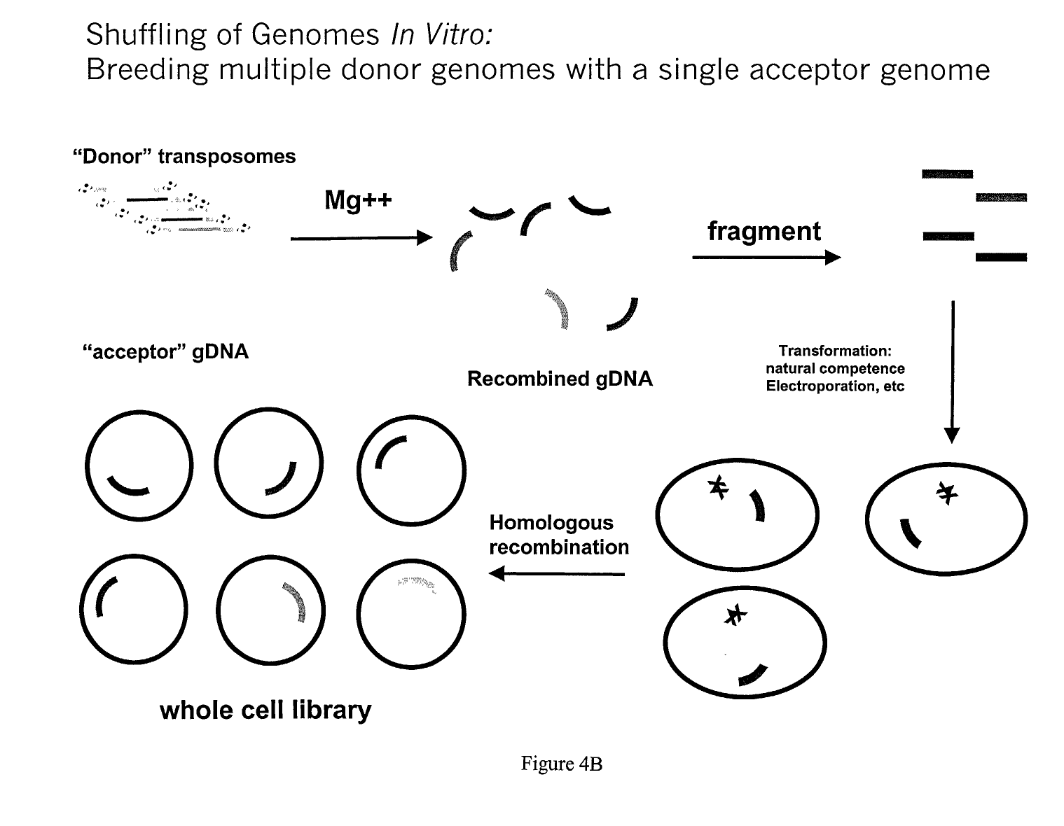

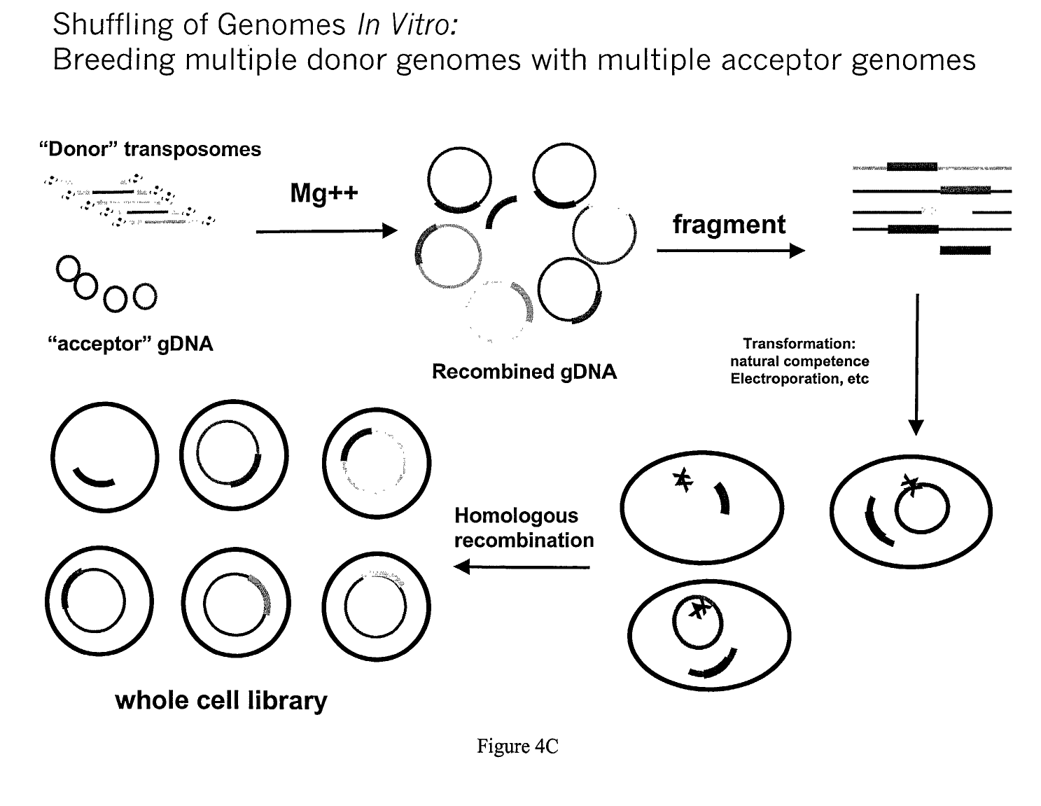

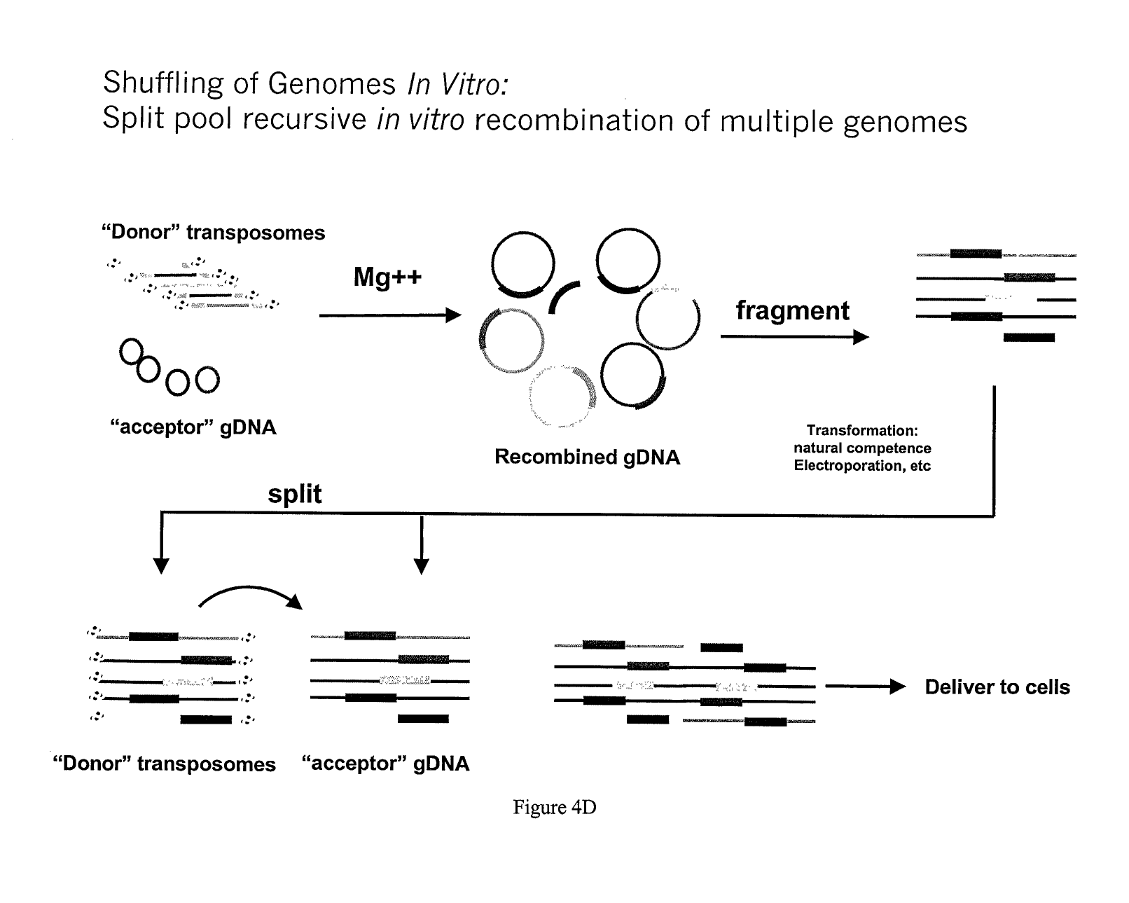

To facilitate the efficient and large scale improvement of industrial strains, high throughput methods requiring reduced operator involvement are preferred. One approach to increasing throughput, while reducing time and effort is by utilizing methods of selection based on the preferential survival of a subset of the population in response to selective pressures in an array of parallel continuous fermentors. A population of recombinant organisms produced by transposon diversification, e.g., shuffling, procedures is used to seed an array of parallel continuous fermentors designated f1 . . . fx (FIG. 3). The fermentors are maintained under desired selection pressures. These selection pressures need not be and most preferably are not at the level that is ultimately desired of the host. Incremental increase in selection pressures are preferred as it prevents complete wash out of the fermentors in response to the severity of the pressure. A special case arises when f1 . . . fx are selecting a single host under incremental increases in the selection pressure (for example temperature) from one fermentor to the other.

The outlet from f11 . . . f1n are fed to another series of parallel continuous fermentors f21 . . . f2n where the corresponding selection pressures are increased by a small amount. A portion of the outlet streams from f21 . . . f2n are recycled respectively to f11 . . . f1n. This process of recycling a cell population back to an environment of lesser intensity of the selection pressure, provides an opportunity for recuperation and expression of desired phenotype. The other portion of the outlet streams from f21 . . . f2n are fed to a column C (WGS) which has been preconditioned for DNA exchange and uptake.

Outlet streams from f21 . . . f2n are fed to WGS as shown in FIG. 3 to foster DNA uptake between different host platforms. Conditions to enhance partial lysis of cultures to release genomic DNA, conditions to stabilize released DNA, and enhance uptake of DNA are maintained in these columns. Other variations include leaking in genomic DNA preparations from other independent experiments or sources which are believed to code for the desired phenotype.

The outlet from the WGS column is fed to another continuous fermentor f31 which is under non selective conditions to provide the opportunity to amplify the genetic diversity created in column WGS.

A portion of the outlet from f31 is distributed equally among fermentors f21 . . . f2n to further seed them with the created diversity and thus continue with the process recursively.

The remaining part of f31 is fed to another continuous fermentor f41 which is under multiple selection pressures so as to enrich for hosts with desired multiple traits or with increased selection pressures. This fermentor is also fed with new media to dilute out strains not meeting the criteria.

Once steady state is reached and a stable population is isolated in f41, the whole process is repeated with increased selection pressures in fermentors f21 . . . f2n. Populations isolated from f41 from the last cycle are used to seed the fermentors f21 . . . f2n in the new cycle.

Alternatively the fermentor f41 is run as a turbidostat where all the phenotypes 1 . . . n are gradually increased towards the desired set points in a combinatorial manner. A portion of the outlet stream from f41 is continuously fed back to the fermentors f21 . . . f2n to further breed diversity.

As shown in FIG. 3, additional genetic diversity can be introduced into the system by spiking in pools of population that have been generated or isolated by other methods independently like transposon mediated genetic diversity, conjugative libraries, shuffled libraries and NTG/UV mutagenized pools, etc., into fermentors f11 . . . f1x or f21 . . . f2x . . .

The above protocol can be easily adapted for phenotypes for which there are no obvious selection pressure. In such cases the continuous fermentors are run under non selective conditions and their outlets are fed into various screening modules (described below in specific applications) that uses one or more criteria to enrich for desired isolates from a population. The enriched populations are fed back to the upstream fermentors and/or fed to the downstream fermentors to continue with the process. In some cases, it will be preferable to miniaturize the process on a “lab-on-a-chip” module (e.g., the LabMicrofluidic device™ high throughput screening system (HTS) by Caliper Technologies Corp., Mountain View, Calif., or the HP/Agilent technologies Bioanalyzer using LabChip™ technology by Caliper Technologies Corp. See, also, calipertech.com) for continuous high throughput generation and selection of microbial diversity for improved phenotypes.

### Application for Evolving Hosts with Improved Process Phenotypes

(a) Faster Growth Rates

Improvements in growth rates of a production host has significant economic advantages. The number of batch fermentations that is typically run during a production cycle can be increased with a host that grows faster. Similarly through-put in continuous production system can be easily increased with a faster growing host. Such improvements in a production host can be achieved by the methodology described here. The selected host (s) is grown in chemostats f11 . . . f1n (FIG. 3) at different dilution rates which are proportional to their respective growth rates. The best available media is selected for this purpose and is kept fixed during the entire process. The choice of the media is often dictated by economic factors and convenience. In chemostats f21 . . . f2n the selection pressure is further tightened by a small amount. To isolate the fastest growing host fermentor f41 is run under even higher stringency of growth rates. In cases where the primary phenotype to be conserved in the host is production of a chemical like amino acids, vitamins, neutraceuticals or a recombinant protein, the fermentor f41 is continuously monitored for productivity as the stringency on growth rate is increased. Populations that grow faster without compromising productivity are recycled to f21 . . . f2n to continue the recursivity of the process.

Most production hosts have been evolved for expression of a primary phenotype in well defined media and process parameters. The genetic material needed to express the desired phenotype under pre-set process conditions is significantly lower than what they generally carry. Significant improvements in product yield, growth rates, and feedstock utilization can be expected by minimizing the genetic make-up (minimal genome) of these production hosts without compromising productivity of the process. For example, attempts have been made to develop identify all essential genes in the mycoplasma genome using transposon mutagenesis. The concept of minimal in this context is in terms of essential genes and not necessarily in terms of minimal physical size. Obtaining a minimal genome in terms of physical size has significant advantages as described above. Methodology described in FIG. 3, in combination with transposition mutagenesis, as described herein and in the references, done iteratively can be used to achieve this goal.

(b) Increased glycolysis (an example for increased feedstock uptake)

The raw material for commercial production of many biochemicals is glucose, fructose, corn starch, etc. An important economic parameter in these processes is the productivity of the process and many metabolic engineering approaches have been made to maximize this feature of a catalyst. Although these approaches are unique to the cases that they are applied to, a common feature of all these bioprocesses is that they all share a common pathway “glycolysis” by which the starting raw material glucose is processed. Ultimately the upper limit on a biotransformation rate using glucose as the feed-stock is limited by how fast a production strain can process glucose through glycolysis.

Although glycolysis is perhaps the most widely studied central metabolism pathway in microbiology, increasing the flux through this pathway (substrate uptake rate) by traditional metabolic engineering approaches have not resulted in any significant improvements. The primary reason for this is lack of significant understanding of how the components of glycolysis interact with cellular physiology and energetics under a given set of production objectives. It is also well known that flux through glycolysis increases significantly under anaerobic conditions compared to aerobic conditions in certain hosts, which suggest that the genetic components and architecture exist in microorganisms to accommodate the phenotype of increased glycolysis. The methodology described here can be applied to evolve a host platform that expresses increased glycolytic rate under a given set of fermentation conditions.

The chosen host is grown in chemostats f11 . . . f1n (FIG. 3) with the selected media and glucose as the limiting substrate. The fluorescent glucose analog 2-NBDG is also added to these chemostats in varying concentrations from one chemostat to the other. 2-NBDG competes with D-Glucose for uptake in a competitive manner and can be monitored by microscopy or single-cell light scattering intensity. The outlet from the chemostats are fed to a cell sorter that enriches for populations that have increased uptake rates for the fluorescent analog. A portion of this enriched populations are recycled to f21 . . . f2x and the rest are fed to the WGS unit (FIG. 3) where genomic breeding continues by one of the methods described herein. The isolation of hosts with increased glucose uptake rates will form the foundation and initial starting point for further evolution of hosts that can channel the increased glucose uptake flux to desirable products like ethanol, lactate, amino acids, isoprenoids, etc. A significant amount of research already exists for engineered hosts that efficiently channel glucose to the above described products.

Similar methodology can be easily adapted for increased uptake of other feedstocks of commercial importance.

(c) Increased TCA cycle (and pentose phosphate cycle)

The tricarboxylic acid cycle is the machinery that microorganisms use to generate energy in the form of NADH by catabolizing carbon sources into CO2. The control of flux through the TCA cycle is complicated and previous attempts to identify rate limiting steps have yielded limited success. Increasing fluxes through the TCA cycle also results in faster NADH production which is beneficial for biotransformations requiring NADH. The methodology described here can be easily adapted to evolve host platforms with increased TCA cycle flux. The flux through TCA cycle, particularly in non growing cells can be calculated from CO2 evolution rates from a chemostat. This measurement can be used to enrich for populations that have increased flux through TCA cycle for a given glucose feed rate and thus can be evolved based on the methodology suggested in FIG. 3.

Similar strategies can be used to create industrial host platforms with the following attributes: increased cofactor recycling rate (cofactor engineering); decreased oxygen radicals; increased efficiency for delivering cytoplasmic molecular oxygen; improved oxidative cytoplasm for increased efficiency of disulfide formation; increased viability in the presence of low pH, organic acids, organic solvents, desiccation, low water content, temperature (high/low), and high osmolarity.

Enrichment of viable populations under above mentioned selection pressures can be achieved using multi-staining flow cytometry as described in literature. This enrichment scheme is integrated to the outlet streams of f21 . . . f2n and thereby enables a continuous enrichment strategy which is beneficial to evolve desired phenotypes.

In addition, the present methods can be used to produce organisms with: increased hydrophobicity (membrane properties) for improved uptake of hydrophobic compounds; improved growth properties under limiting dissolved oxygen concentrations in the fermentor; increased or sustained metabolism in the presence of high end product concentration; and organisms that utilize cheaper sources of reducing equivalents like ethanol, methanol, alkanes, etc., with high efficiency to drive biotransformations (e.g., that require reducing power).

### MOLECULAR BIOLOGY

General texts which describe molecular biological techniques useful herein, including the use of vectors, promoters and many other relevant topics related to, e.g., the cloning and expression of transposable elements, transposons, insertion sequences, and their components, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). Similarly, examples of techniques sufficient to direct persons of skill through in vitro amplification methods, including the polymerase chain reaction (PCR) the ligase chain reaction (LCR), Qβ-replicase amplification and other RNA polymerase mediated techniques (e.g., NASBA), e.g., for the production of the homologous nucleic acids of the invention are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

The present invention also relates to host cells and organisms which are transformed with vectors of the invention, and the production of polypeptides of the invention, e.g., transposases, exogenous DNAs incorporated into transposable elements or insertion sequences, by recombinant techniques. Host cells are genetically engineered (i.e., transformed, transduced or transfected) with the vectors of this invention, which can be, for example, a cloning vector or an expression vector. The vector can be, for example, in the form of a plasmid, a virus, a naked polynucleotide, or a conjugated polynucleotide. The vectors are introduced into cells by standard methods including electroporation (From et al. (1985) Proc. Natl. Acad. Sci. USA 82:5824, infection by viral vectors such as cauliflower mosaic virus (CaMV) (Hohn et al. (1982) Molecular Biology of Plant Tumors (Academic Press, New York) pp. 549-560; Howell, U.S. Pat. No. 4,407,956), high velocity ballistic penetration by small particles with the nucleic acid either within the matrix of small beads or particles, or on the surface (Klein et al. (1987) Nature 327:70-73), also, especially in the case of plant cells by the use of pollen as vector (WO 85/01856), or use of Agrobacterium tumefaciens or A. rhizogenes carrying a T-DNA plasmid in which DNA fragments, e.g., including transposable elements, are cloned. The T-DNA plasrnid is transmitted to plant cells upon infection by Agrobacterium tumefaciens, and a portion is stably integrated into the plant genome (Horsch et al. (1984) Science 233: 496-498; Fraley et al. (1983) Proc. Natl. Acad. Sci. USA 80: 4803).

The engineered host cells can be cultured in conventional nutrient media modified as appropriate for such activities as, for example, activating promoters or selecting transformants. Where appropriate cells can be optionally cultured into transgenic organisms. For example, plant regeneration from cultured protoplasts is described in Evans et al.(1983) “Protoplast Isolation and Culture,” Handbook of Plant Cell Cultures 1:124-176 (MacMillan Publishing Co., New York); Davey (1983) “Recent Developments in the Culture and Regeneration of Plant Protoplasts,” Protoplasts pp. 12-29, (Birkhauser, Basel); Dale (1983) “Protoplast Culture and Plant Regeneration of Cereals and Other Recalcitrant Crops,” Protoplasts pp. 31-41, (Birkhauser, Basel); Binding (1985) “Regeneration of Plants,” Plant Protoplasts pp. 21-73, (CRC Press, Boca Raton).

The present invention also relates to the production of transgenic organisms, which can be bacteria, yeast, fungi, or plants. A thorough discussion of techniques relevant to bacteria, unicellular eukaryotes and cell culture can be found in references enumerated above and are briefly outlined as follows. Several well-known methods of introducing target nucleic acids into bacterial cells are available, any of which can be used in the present invention. These include: fusion of the recipient cells with bacterial protoplasts containing the DNA, electroporation, projectile bombardment, and infection with viral vectors (discussed further, below), etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAprep™ from Qiagen). The isolated and purified plasmids are then further manipulated to produce other plasmids, used to transfect plant cells or incorporated into Agrobacterium tumefaciens related vectors to infect plants. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith (1979) Gene 8:81; Roberts et al. (1987) Nature 328:731; Schneideret al. (1995) Protein Expr. Purif. 6435:10; Ausubel, Sambrook, Berger (all supra). A catalogue of Bacteria and Bacteriophages useful for cloning is provided, e.g., by the ATCC, e.g., The ATCC Catalogue of Bacteria and Bacteriophage (1992) Ghema et al. (eds) published by the ATCC. Additional basic procedures for sequencing, cloning and other aspects of molecular biology and underlying theoretical considerations are also found in Watson et al. (1992) Recombinant DNA (Second Edition) Scientific American Books, NY.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method for producing one or more transposable element component with a desired property, the method comprising:
i) providing a population of polynucleotide segments comprising at least one transposable element or subportion of a transposable element;
ii) recombining the polynucleotide segments one or more times, thereby producing a library of recombinant transposable element components;
iii) identifying at least one recombinant transposable element component with a desired property;
iv) optionally repeating steps (i) through (iii) at least one additional time.

2. The method of claim 1, further comprising recovering the transposable element or a subportion thereof by a polymerase chain reaction (PCR), ligase chain reaction (LCR), Qβ-replicase amplification, NASBA or cloning.

3. The method of claim 1, comprising providing a population of polynucleotide segments comprising at least one component of a transposon or insertion sequence (IS) element or a subportion of a component of a transposon or IS element.

4. The method of claim 3, wherein the at least one component of a transposable element comprises an inverted repeat or a transposase of a transposon or an IS element.

5. The method of claim 3, wherein the transposon or IS element comprises a mini-transposon or a mini-IS element.

6. The method of claim 1, wherein at least one polynucleotide segment comprises a transposable element or a subportion of a transposable element of a bacterium, a fungus, a plant or an animal.

7. The method of claim 1, wherein the transposable element comprises a Class I or a Class II transposable element.

8. The method of claim 7, wherein the Class I transposable element comprises a retrotransposon, a retroposon or a SINE-like element.

9. The method of claim 8, wherein the Class I transposable element comprises a Ty-1 family transposon, a Copia family transposon, or a gypsy family transposon.

10. The method of claim 7, wherein the Class II transposable element comprises a Fot1/Pogo family transposon, a Tc1/Mariner family transposon,

11. The method of claim 7, wherein the transposable element is selected from the group consisting of TN3, TN5, TN10, TN917, ISS1, TN5990, Ty1, Ty2, Ty3, and mariner.

12. The method of claim 1, comprising recombining the polynucleotide segments in vitro, in vivo, or in silico.

13. The method of claim 1, wherein the desired property is selected from one or more of altered specificity of integration, host adaptation, altered cofactor specificity, increased or decreased recombinase activity, increased or decreased transposase activity, increased or decreased recombinase specificity, increased or decreased transposase specificity, increased or decreased size of exogenous DNA transposed, increased or decreased copy number, increased or decreased efficiency of transposition, increased or decreased preference for episomal targeting, increased or decreased preference for chromosomal targeting, increased efficiency of integration into non-supercoiled DNA, and increased efficiency of in vitro transposition.

14. The method of claim 1, wherein the identifying of step (iii) comprises screening or selecting at least one transposable element with a desired property.

15. The method of claim 14, comprising identifying at least one transposable element that mediates transposition in vitro with greater efficiency when compared to a parental transposable element, the method comprising:
providing a plurality of in vitro transposition reactions, which in vitro transposition reactions comprise:
(a) a transposase;
(b) a donor polynucleotide comprising at least one inverted repeat; and
(c) a target polynucleotide
incubating the plurality of in vitro transposition reactions under conditions permissive for in vitro transposition; and
identifying at least one in vitro transposition reaction that occurs with greater efficiency than an in vitro transposition reaction mediated by a parental transposable element.

16. The method of claim 15, comprising providing in vitro transposition reactions comprising transposomes, which transposomes comprise the transposase and the donor polynucleotide.

17. The method of claim 14, comprising identifying at least one transposable element that transposes with increased efficiency in a specified host cell when compared with a wild type transposable element, the method comprising:
(a) introducing a plurality of transposable elements, which transposable elements differ by at least one nucleotide, into a population of host cells;
(b) selecting at least one host cell that has integrated the transposable element into a chromosome or episome.

18. The method of claim 17, the transposable element comprising in the direction of transcription (a) a polynucleotide comprising a transcription regulatory sequence; (b) a 5′ splice donor site; (c) a first inverted repeat; (d) a 3′ splice acceptor site; (e) a polynucleotide encoding a transposase; (f) a polynucleotide encoding a selectable marker; and (g) a second inverted repeat.

19. The method of claim 18, which transposable element transiently expresses the transposase.

20. The method of claim 17, comprising selecting at least one host cell that expresses a sufficient level of a selectable marker encoded by the transposable element.

21. The method of claim 17, further comprising recovering the transposable element.

22. The method of claim 17, the population of host cells comprising mammalian cells.

23. The method of claim 17, wherein the transposable element comprises a Mariner transposase, and wherein the inverted repeats comprise Mariner inverted repeats.

24. The method of claim 23, wherein the Mariner transposase comprises a Himar1 transposase.

25. The method of claim 17, wherein the selectable marker comprises drug resistance.

26. The method of claim 25, wherein the antibiotic resistance is selected from among neomycin resistance, kanamycin resistance.

27. The method of claim 1, wherein the transposable element comprises a recombinant vector.

28. The method of claim 27, wherein the recombinant vector is a delivery vector comprising (a) an origin of replication active in at least one cloning host; (b) a conditional origin of replication active in at least one target cell; (c) at least one screenable or selectable marker; (d) a mini-transposon comprising a first inverted repeat and a second inverted repeat, which inverted repeats flank a multicloning site (MCS); and (e) a transposase operably linked to a promoter active in at least one target cell.

29. The method of claim 28, wherein the transposase is in close proximity to at least one end of the minitransposon.

30. The recombinant delivery vector of claim 28.

31. The recombinant delivery vector of claim 30, the origin of replication (a) comprising an origin of replication selected from among a ColE1 origin, a pACYC origin, a p15A origin, an RK4 origin, an RK6 origin, a pCM595 origin, a pSa origin, a pUB110 origin, a pE194 origin, a pG+ origin, a 2 micron circle origin, and an artificial chromosome origin.

32. The recombinant delivery vector of claim 30, the conditional origin of replication (b) comprising a temperature sensitive origin of replication selected from among a gram negative origin, a pSA3 origin, a pE194tm origin, and a pG+tm origin.

33. The recombinant delivery vector of claim 30, the at least one selectable or screenable marker (c) comprising antibiotic resistance, conferred prototrophy, or toxicity resistance.

34. The recombinant delivery vector of claim 31, wherein the antibiotic resistance marker comprises kanamycin resistance, ampicillin resistance, macolidelincosamine-streptogramin (MLS) resistance, apramycin resistance, spiramycin resistance, hygromycin resistance, chloramphenicol resistance, or tetracycline resistance.

35. The recombinant delivery vector of claim 30, wherein the mini-transposon (d) is derived from a transposon or insertion sequence of table 1.

36. The recombinant delivery vector of claim 30, the transposase (e) comprising a naturally occuring transposase or a transposase derived by one or more directed evolution method.

37. The recombinant delivery vector of claim 36, wherein the promoter is selected from an endogenous promoter of a target cell.

38. The recombinant delivery vector of claim 30, comprising in the order of transcription: a polynucleotide encoding a transposase operably linked to a promoter functional in a target cell, a mini IS element, which mini-IS element comprises a first IS inverted repeat and a second IS inverted repeat, which first and second IS inverted repeats flank a multicloning site, a first origin of replication functional in cloning host, a first selectable marker, a second selectable marker, and a second origin of replication, which origin of replication is temperature sensitive.

39. The recombinant delivery vector of claim 38, wherein the transposase comprises: a transposon or IS element int encoding sequence and a transposon or IS element xis encoding sequence.

40. The recombinant delivery vector of claim 38, wherein the first or second selectable marker comprises a drug resistance marker selected from: ampicillin resistance, kanamycin resistance, chloramphenicol resistance, neomycin resistance, tetracycline resistance, erythromycin resistance and G418 resistance.

41. The recombinant delivery vector of claim 38, wherein the first and second selectable markers comprise two alternative markers selected from: ampicillin resistance, kanamycin resistance, chloramphenicol resistance, neomycin resistance, tetracycline resistance, erythromycin resistance and G418 resistance.

42. The recombinant delivery vector of claim 38, wherein the second origin of replication comprises a thermosensitive replicon of pG+.

43. The recombinant delivery vector of claim 38, wherein the vector comprises a pTNWGS vector.

44. A transposable element with a desired property produced by the method of claim 1.

45. The transposable element of claim 44, wherein the desired property is selected from one or more of altered specificity of integration, host adaptation, increased or decreased recombinase activity, increased or decreased transposase activity, increased or decreased recombinase specificity, increased or decreased transposase specificity, increased or decreased size of exogenous DNA transposed, increased or decreased copy number, increased or decreased efficiency of transposition, increased or decreased preference for episomal targeting, increased or decreased preference for chromosomal targeting, increased efficiency of integration into non-supercoiled DNA, and increased efficiency of in vitro transposition.

46. The transposable element of claim 44, which transposable element catalyzes in vitro transposition more efficiently than a parental transposable element.

47. The transposable element of claim 44, which transposable element integrates into a specified host cell with increased efficiency when compared to a wild type transposable element.

48. A component of a transposable element with a desired property produced by the method of claim 1.

49. The transposable element component of claim 48, wherein the component comprises a transposase, a recombinase or an integrase.

50. The transposable element component of claim 49, comprising a transposase that catalyzes in vitro transposition more efficiently than a parental transposase.

51. The transposable element component of claim 48, wherein the component comprises an inverted repeat.

52. A method for producing a transposase that efficiently catalyzes in vitro transposition, the method comprising:
i) providing a population of polynucleotide segments encoding at least one transposase or subportion of a transposase;
ii) recombining the polynucleotide segments one or more times, thereby producing a library of recombinant polynucleotides encoding variant transposases;
iii) identifying at least one recombinant polynucleotide encoding a transposase that efficiently catalyzes in vitro transposition.

53. The method of claim 52, comprising identifying the at least one recombinant polynucleotide encoding a transposase that efficiently catalyzes in vitro transposition by:
a) providing a plurality of in vitro transposition reactions, which in vitro transposition reactions comprise a transposase encoded by the recombinant polynucleotide, a donor polynucleotide comprising at least one inverted repeat, and a target polynucleotide;
b) incubating the plurality of in vitro transposition reactions under conditions permissive for in vitro transposition; and
c) identifying at least one in vitro transposition reaction that occurs with greater efficiency than an in vitro transposition reaction mediated by a parental transposase.

54. A transposase produced by the method of claim 52.

55. The transposase of claim 54, wherein the transposase is selected from among transposases derived by a directed evolution process from at least one transposase of TN5, TN10, TN917, ISS1, TN5990, Ty1, Ty2, Ty3, or mariner.

56. A reaction mix or a cell comprising the transposase of claim 54.

57. A method for generating diversity in a population of nucleic acids, the method comprising: contacting at least one recombinant transposable element or recombinant transposable element component, and a plurality of subject nucleic acids under conditions permissive for transposition.

58. The method of claim 57, wherein the recombinant transposable element or recombinant transposable element component is produced by one or more diversity generating procedure.

59. The method of claim 57, wherein the recombinant transposable element or recombinant transposable element component is produced by recursive recombination.

60. The method of claim 57, further comprising identifying at least one altered subject nucleic acid.

61. The method of claim 57, comprising contacting the recombinant transposable element or recombinant transposable element component and the subject nucleic acids in vivo.

62. The method of claim 61, wherein the recombinant transposable element component comprises a recombinant transposase.

63. The method of claim 62, comprising introducing a transposome, which transposome comprises the recombinant transposase bound to a donor nucleic acid, which donor nucleic acid comprises sequences recognized by the recombinant transposase, into a cell, thereby contacting the recombinant transposable element component and the subject nucleic acids.

64. The method of claim 63, comprising introducing the transposome into the cell by electroporation.

65. The method of claim 57, comprising contacting the transposable element or transposable element component and the subject nucleic acids in vitro.

66. The method of claim 65, wherein the recombinant transposable element component comprises a recombinant transposase.

67. The method of claim 65, comprising contacting the subject nucleic acids with a transposome, which transposome comprises the shuffled transposase bound to a donor nucleic acid, which donor nucleic acid comprises sequences recognized by the shuffled transposase, in an acellular reaction mix.

68. A method for generating diversity in a population of nucleic acids, the method comprising:
i) providing a plurality of transposomes, which transposomes comprise a library of donor nucleic acids, and a population of acceptor nucleic acids in vitro;
ii) recombining the donor nucleic acids and the acceptor nucleic acids to produce a library of recombinant nucleic acids.

69. The method of claim 68, comprising recombining the donor nucleic acids and the acceptor nucleic acids in the presence of magnesium ions.

70. The method of claim 68, comprising providing the transposome by combining a plurality of donor nucleic acid molecules, which donor nucleic acid molecules comprise transposable element recognition sequences and a plurality of transposase molecules, which transposase molecules bind the transposable element recognition sequences.

71. The method of claim 70, wherein the donor nucleic acids comprising transposable element recognition sequences are produced by cloning genomic DNA fragments into a mini-transposon or mini-insertion sequence.

72. The method of claim 71, wherein the genomic DNA fragments are restriction enzyme fragments.

73. The method of claim 71, wherein the mini-transposon comprises a Tn5 mini-transposon.

74. The method of claim 71, wherein the mini-transposon comprises a mariner transposon.

75. The method of claim 68, wherein one or more of the donor or acceptor nucleic acids are derived from a plurality of organisms.

76. The method of claim 68, further providing at least one population of additional nucleic acids.

77. The method of claim 76, the population of additional nucleic acids comprising one or more of a promoter, a regulatory element, a terminator sequence, an antiterminator sequence, a sequence comprising a start codon, a sequence comprising a stop codon, a library of recombinant genes, a gene of interest, or an IS element.

78. The method of claim 68, further comprising repeating the recombination of steps i) and ii) by providing transposomes comprising the library of recombinant nucleic acids or a subportion thereof.

79. The method of claim 68, further comprising, introducing the library of recombinant nucleic acids or a subportion thereof into a population of cells and identifying at least one cell with a desired property.

80. The method of claim 79, comprising introducing the library of recombinant nucleic acids or a subportion thereof into the population of cells by a delivery method comprising natural competence, conjugation, transformation, electroporation, or infection with bacteriophage.

81. A method for identifying a chromosomal locus, which chromosomal locus exhibits a desired level of gene expression, the method comprising:
i) transfecting a plurality of host cells expressing a transposase with a vector comprising, in the direction of transcription: (a) a first inverted repeat; (b) a promoter; (c) a site specific recombinase recognition site; (d) a polynucleotide encoding a first screenable or selectable marker; (e) a polynucleotide encoding a second screenable or selectable marker; and (f) a second inverted repeat;
ii) identifying at least one host cell that expresses a sufficient level of at least one selectable marker, which selectable marker is encoded by the first or second visible or selectable marker, to survive selection, thereby identifying at least one host cell that has integrated the vector into a chromosome; and
iii) identifying at least one host cell expressing at least one screenable or selectable marker at a desired level, thereby identifying a chromosomal locus exhibiting a desired level of gene expression.

82. The method of claim 81, wherein the vector further comprises a polynucleotide encoding the transposase operably linked to a promoter active in the host cells.

83. The method of claim 81, further comprising integrating a polynucleotide sequence of interest into the identified chromosomal locus to generate at least one integrant.

84. The method of claim 82, further comprising identifying at least one integrant with a desired level of expression.

85. The method of claim 81, wherein the inverted repeats comprise transposable element inverted repeats.

86. The method of claim 85, wherein the inverted repeats comprise Mariner inverted repeats.

87. The method of claim 81, wherein the site specific recombinase recognition site comprises a loxP site.

88. The method of claim 81, wherein the promoter comprises a cytomegalovirus (CMV) promoter.

89. The method of claim 81, wherein the first or second screenable or selectable marker is a selectable marker selected from among: antibiotic resistance, herbicide resistance, neomycin resistance, kanamycin resistance.

90. The method of claim 81, wherein the fist or second screenable or selectable marker is a visible marker selected from among: green fluorescent protein (GFP), luciferase, β-galactosidase, β-glucuronidase, alkaline phosphatase.

91. The method of claim 81, wherein the first screenable or selectable marker comprises a visible marker and the second screenable or selectable marker comprises a selectable marker.

92. The method of claim 91, wherein the visible marker is GFP and the selectable marker is neomycin resistance.

93. The method of claim 81, the plurality of cells comprising bacterial, fungal, animal or plant cells.

94. The method of claim 81, wherein the transposase is encoded by a chromosomal sequence.

95. The method of claim 81, wherein the transposase is encoded by a polynucleotide comprising an additional vector.

96. The method of claim 95, wherein the additional vector comprises an episomal vector.

97. The method of claim 95, wherein the vector comprises a chromosomally integrated vector.

98. The method of claim 95, comprising expressing the transposase transiently.

99. The method of claim 98, comprising expressing the transposase inducibly.

100. The method of claim 81, comprising expressing a Mariner transposase.

101. The method of claim 100, wherein the transposase comprises an artificially evolved transposase, which artificially evolved transposase has at least one property which differs from a parental transposase from which it is derived by directed evolution.

102. The method of claim 101, wherein the at least one property which differs from the parental transposase is selected from among: sequence specificity, activity level, species selectivity, allostery, and control.

103. A vector comprising (a) a first inverted repeat; (b) a promoter; (c) a site specific recombinase recognition site; (d) a polynucleotide encoding a first screenable or selectable marker; (e) a polynucleotide encoding a second screenable or selectable marker; and (f) a second inverted repeat.

104. The vector of claim 103, wherein the inverted repeats comprise transposable element inverted repeats.

105. The vector of claim 104, wherein the inverted repeats comprise Mariner inverted repeats.

106. The vector of claim 103, wherein the site specific recombinase recognition site is a loxP site.

107. The vector of claim 103, wherein the promoter comprises a cytomegalovirus (CMV) promoter.

108. The vector of claim 103, wherein the first or second screenable or selectable marker is a selectable marker selected from among: antibiotic resistance, herbicide resistance, neomycin resistance, kanamycin resistance.

109. The vector of claim 103, wherein the first or second screenable or selectable marker is a visible marker selected from among: green fluorescent protein (GFP), luciferase, β-galactosidase, β-glucuronidase, alkaline phosphatase.

110. The vector of claim 103, wherein the first screenable or selectable marker comprises a visible marker and the second visible or selectable marker comprises a selectable marker.

111. The vector of claim 103, wherein the visible marker is GFP and the selectable marker is neomycin resistance.

112. A vector comprising in the direction of transcription: (a) a polynucleotide comprising a transcription regulatory sequence; (b) a 5′ splice donor site; (c) a first inverted repeat; (d) a 3′ splice acceptor site; (e) a polynucleotide encoding a transposase; (f) a polynucleotide encoding a selectable marker; and (g) a second inverted repeat.

113. The vector of claim 103, wherein the first and second inverted repeat comprises Mariner inverted repeats.

114. The vector of claim 103, wherein the transposase comprises a Mariner transposase.

115. The vector of claim 103, wherein the first and second inverted repeats comprise Mariner inverted repeats, and the transposase comprises a Mariner transposase.
